SEQUENCE LISTING (19 sequences)

SEQ ID NO: 1 - DNA, 1377 bp, Homo sapiens
agcatcctgagtaatgagtggcctgggccggagcaggcgaggtggccggagccgtgtggaccaggaggagcgctttccacagggcctgtggacgggggtggctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagaagaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagtggccttggcattgtgtgtgtgcctgcccaggaggggggcccaggtgcataaatgggggtcagggagggaaaggaggagggagagagatggagaggaggggagagagaaagagaggtggggagaggggagagagatatgaggagagagagacagaggaggcagaaagggagagaaacagaggagacagagagggagagagagacagagggagagagagacagaggggaagagaggcagagagggaaagaggcagagaaggaaagagacaggcagagaaggagagaggcagagagggagagaggcagagagggagagaggcagagagacagagagggagagagggacagagagagatagagcaggaggtcggggcactctgagtcccagttcccagtgcagctgtaggtcgtcatcacctaaccacacgtgcaataaagtcctcgtgcctgctgctcacagcccccgagagcccctcctcctggagaataaaacctttggcagctgcccttcctcaaaaaaaaaaaaaaaaaaaa

SEQ ID NO: 2 - DNA, 1377 bp, Homo sapiens
ttttttttttttttttttttgaggaagggcagctgccaaaggttttattctccaggaggaggggctctcgggggctgtgagcagcaggcacgaggactttattgcacgtgtggttaggtgatgacgacctacagctgcactgggaactgggactcagagtgccccgacctcctgctctatctctctctgtccctctctccctctctgtctctctgcctctctccctctctgcctctctccctctctgcctctctccttctctgcctgtctctttccttctctgcctctttccctctctgcctctcttcccctctgtctctctctccctctgtctctctctccctctctgtctcctctgtttctctccctttctgcctcctctgtctctctctcctcatatctctctcccctctccccacctctctttctctctcccctcctctccatctctctccctcctcctttccctccctgacccccatttatgcacctgggcccccctcctgggcaggcacacacacaatgccaaggccactgtctgggatgtgtgggcaaggctgcaggactgtggtcctggtgtggcacccccaccttccagcacaagtggggtcgggggtcccaggcgtgactgcgctctcctgcgtgggcgccctgcactcagggaagcagaagctgcaggtctccactggctcgggggatgtgctcacagggctgccggcttccatcgcgtgatcctgggaagacttggccggactttgacggggccttgagcggggctggcaggagcagggatcccccctcttcttgaggaagcaggccaccgccaccaggaagcagcagaggacggcacacaggcagagccccagcgtgctgtagaccagggccacctgatctgcactcagcttcagccccgggagagctggacttgcttctgagcctctgtgctccaatccttggtaccttcccgagttgtctgaattgttttcaacttctccactccgctgtctcctgagctctggtggaaggttcactgggctcctgagcttgttctcacagaagtatgcacattgcttagggtgctgtccacagatggaggcacagctgatgcagtccctcaggagatggtcatagaacttgccttgctccttgcggcagctgagtgacctgcagaaggctgcacaggtgcgctggctctgatggttgcaaatggttttgcaggacatgcaggtacccagcagaggatcccagtactgctcttcggggcaggatctcatagccacccccgtccacaggccctgtggaaagcgctcctcctggtccacacggctccggccacctcgcctgctccggcccaggccactcattactcaggatgct

SEQ ID NO: 3 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 4 - DNA, 995 bp, Homo sapiens
aagactcaaacttagaaacttgaattagatgtggtattcaaatccttacgtgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaagtctttttttttcctga

SEQ ID NO: 5 - DNA, 995 bp, Homo sapiens
tcaggaaaaaaaaagactttaatcatgaaaccaaggaagtttctaccattaagctcccaacagtaacctagagaaatatatctaacataaagagtttccacagttagaggacaaaaagctgtatcggcaactgaaaaatacagtcatcctaaagagatctgacacatcatctattctgacaaaagatttttaaagtggcactgctcgagtcgaaatggttaattacctagcagaaattgatttctctatctccgtagcactcaaagcagctggcaggctcttgcaatagtcattcgttttcgtggtgacaagaatggttgcgccttcctccatagctgggagtggaaagcaatggtcagagtcgaccttcggtttgctcttgatgcagtcttcacaggtgcattcttccaccgtgtactcgaggcctctcggaagaataatttcatcaccagtcctgctcttttccaggtcaatgttagccatgcccaggagacctgatcctgtgtttttaaactcgtcctttaatggttcagagcttatcttccttagcaaaaacattagcacgaaaactgccaaagaaattattaagctcagtcccaaacaggtccagagaatcgcattcgttcctttcactgaattggtcacacttgcattacaataacgctgacatgttagaggaggagtattagaagaacatcgaagttgacaaggtatgcaagcatgcaacaaactgtcaaaatattcattttgggagcactgcccagccatctgcaacatgatcacaaaaagaaaacaagggagctacagaaagaacagcctggaaggacaagtaatatctctacaagaattccagagcaaatgcagcaagagcagctagaatgttgagaacaatgaacttcgcctgcttcgtgggttcttacgggggctgtctgtgtcttcgcggcacgtaaggatttgaataccacatctaattcaagtttctaagtttgagtctt

SEQ ID NO: 6 - protein, 184 aa, Homo sapiens [sequence omitted]

SEQ ID NO: 7 - DNA, 858 bp, Homo sapiens
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcttggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtga

SEQ ID NO: 8 - DNA, 858 bp, Homo sapiens
tcacagcagtttcaatgcaccaaaaaatgtgacatctccatccagtgatatttgtgcattttctcttggtattgcaagttggagttcatctccttcttccagttttgcaatgccagctgaatagcaggaattattgggtagtgtttcaggcatattttgaatacatcgaaacaaagtcaccagactcaattcatccccaaagacatggaccttcttcctctgaattagatgtcccatggcgtaggtcttatcagtatataaaacctgaccatatataaaaaagtaaccagtttctttgaccaatattttattctctttttcttctagggcacttccccttttaaagctgagaagccatggaacaaatgtgtaagatcctttttgtatagttggtgtttcactgtctgcaatcagttgcaagcagtcttgagtgactgtttcttctggaccctgaacggcacgcttatttctgctgttctgactggagttgccttctcctggagctggtggttcaaagattttcagtcccgcggtgacagctggagcttcctccaagccggccttgggggctcctgctcctgctggcagcttctccgcgtggtggccctgcagctctgcccggaggctggccaggtccccttgcagggcggccacctggtagaaagacaccaccgtgaggcagcaagacagcagtgccagcagcaaggttgcagccagcagctttccgtctttggaggatcggacagaggggctttccttccgtgggaggatggaaacacactccttcagtttcatttcttctcttttcttaaggcaagaagtaaggcgtgactgctccctttctgtggagtcatccat

SEQ ID NO: 9 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 10 - DNA, 1348 bp, Homo sapiens
ggtacgaggcttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctta

SEQ ID NO: 11 - DNA, 1348 bp, Homo sapiens
taagatggtggtgctgttctggatgcttccagtgggccccgaccaggtctgaacaatgcctggcgcccgtcccccgcccctcatctacacacacgcaagaattcggagctccatggggaacagaagcaagatatccgtaaaatcaaagtctagggggtgggaatgaaaagggaaaagtgaggaacggggagccaaacccaggaagatgcctctgttcctgcacattccctctcctttatatactcagctcttggctgtctccagtatgtacccaccctggtcttccaagctgggagccactttttataacacaatcacagtttcacaaaccccaggaaggttccatgtggagagaggttaagtttcgcccttgcccggggaattatgacactcagaatatccccttggtgtaaatggaagacacctgcgctatagcagctgttgtaggcccggtccgggtgggagggcatacttcttatacatcggaatagagtctcctgccttccttggccttctcgagacaccacctgacccatggtgaaagtcacgtcttgaaacaggacctggctatacagcagataaactccagcatcctggattcggacaccatatccttgggcctgtaggcctctcccacgcctaagagctggttgccacatcacctctgtcacatcggagtcatccttggaggtggcgttaatgggaaccaggtgcaggacagagtgctgcttcttctgtttttgggtgagcactgctctccttttccgggatctctccccattctcccaggcttccagggcatcggaactctgctccgggagactctgccagggatacccttccccattctgggaggggcctcctgtcccctgcagccggctcacctctctcctgaggctctgcagctctgtttgttgggtcagcagagccatggcacaagccacggcccccagagctgccccccaactcaaccagagggcaactgagagtgccggctctctgactgggccccccatgttgcctggaggccctttgggggctagcaagaaaggagatgaggctggcatgagctggggaccctgccaacagctgtggtttcaagagtggggtagcaaggaggtggcggggcgggtaagggtacgggcagtggtgcagaagggaagaaggttgttacgcaaggagaaataaaaaggaacttgaaaataaaaaggagggaggaggaaagcaagctaagggtactgttagtgctcctggcactccgtcgtggggccagcgttgccttgagaccctccaccctccctcagcctcaggagaattaggttccagtccctctaggaagcctcgtacc

SEQ ID NO: 12 - protein, 250 aa, Homo sapiens [sequence omitted]

SEQ ID NO: 13 - DNA, 1239 bp, Homo sapiens
agcatcctgagtaatgagtggcctgggccggagcaggcgaggtggccggagccgtgtggaccaggaggagcgctggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagaagaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagtggccttggcattgtgtgtgtgcctgcccaggaggggggcccaggtgcataaatgggggtcagggagggaaaggaggagggagagagatggagaggaggggagagagaaagagaggtggggagaggggagagagatatgaggagagagagacagaggaggcagaaagggagagaaacagaggagacagagagggagagagagacagagggagagagagacagaggggaagagaggcagagagggaaagaggcagagaaggaaagagacaggcagagaaggagagaggcagagagggagagaggcagagagggagagaggcagagagacagagagggagagagggacagagagagatagagcaggaggtcggggcactctgagtcccagttcccagtgcagctgtaggtcgtcatcacctaaccacacgtgcaataaagtcctcgtgcctgctgctcacagcccccgagagcccctcctcctggagaataaaacctttggcagctgcccttcctcaaaaaaaaaaaaaaaaaaaa

SEQ ID NO: 14 - protein, 247 aa, Homo sapiens
MSGLGRSRRGGRSRVDQEERWSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 15 - DNA, 595 bp, Homo sapiens
cgtcggcaccatgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatagcagggagccggcaggaggtggcccctgccc

SEQ ID NO: 16 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 17 - DNA, 1882 bp, Mus musculus
atgggcgccaggagactccgggtccgaagccagaggagccgggacagctcggtgcccacccagtgcaatcagaccgagtgcttcgaccctctggtgagaaactgcgtgtcctgtgagctcttccacacgccggacactggacatacaagcagcctggagcctgggacagctctgcagcctcaggagggctccgcgctgagacccgacgtggcgctgctcgtcggtgcccccgcactcctgggactgatactggcgctgaccctggtgggtctagtgagtctggtgagctggaggtggcgtcaacagctcaggacggcctccccagacacttcagaaggagtccagcaagagtccctggaaaatgtctttgtaccctcctcagaaacccctcatgcctcagctcctacctggcctccgctcaaagaagatgcagacagcgccctgccacgccacagcgtcccggtgcccgccacagaactgggctccaccgagctggtgaccaccaagacagctggcccagagcaatagcagcagtggaggctggaacccagggatctctactgggcttgtggacttcacccaacagcttgggaaagaacttggcccttcagtgacggagtcctttgcctggggggcgaacccggcagaaccagacactacaggccacatgagattgcttttgtgttagctcttgacttgagaacgttccatttctgagatggtttttaagcctgtgtgccttcagatggttggatagacttgagggttgcatatttaatctctgtagtgagtcggagactggaaacttaatctcgttctaaaaattttggattactgggctggaggtatggctcagcagttcggtttgtgtgctgttctagccgaggactccagttgttcagcttcccggaactcagatctggcagcttaagaccacctgtcactccagcccctggaacatccttgcctccaaaggcaccagcactgatttgctctagagcacacacacacacacacacacacacacacacacacacacacacatatgcatgcatgcacacttaaaaatgtcaaaattagcggctggagaaattcatggtcaacagcgcttactgtgattccagaggatgagagtttgattcccagaatgcactgcgggtggctcattactgagcataacttttgcttcaggggacctgatgcctctggacttcatgggcatctgtattcacgtgcacatcctacacacacacacacacacacacacagacatacacacacacacactcttttacaaatgataaaatataagatgggcatggtggtacacacctttaatcccaacattggggaagcaaaggcaggcaggtaaatgagttggaggccatcctggtctacatagcaagttccaggctaaccagagctaaatggtgagaccaagtctcaaaataatactcccccccccaaaaaaaaaacttttaaattttgatttttttcttttattattattttttatattaatttcatggtgtttagaagtggtatacttagatggtgactaagaggaggtaaagccatcaggactgagcccctaacatacaaggagaaagcagagacaatgaacacgcccctctcctgctgtgtgccagctctggaccaccagccagagggcaatcatcagatgtgggccctagaaccttcagagccgaaagctaaatcaatctcatttctttgtaaagctatttagccttaggtgttttgttacggtgatataaaatggactaacacaggcactatgagtaagaagcttttctttgagctgggaaaggtactgttaaaccaaaattaatctgaataaaaaaaggctaaggggaagacactt

SEQ ID NO: 18 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 19 - protein, 265 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRTAPPREG

(19) United States
(12) Patent Application Publication
(10) Pub. No.: US 2005/0070689 A1
(43) Pub. Date: Mar. 31, 2005

(54) Taci and br3 polypeptides and uses thereof

(75) Inventors: Vishva Dixit, Los Altos, CA (US); Iqbal Grewal, Fremont, CA (US); John Ridgway, San Francisco, CA (US); Minhong Yan, Burlingame, CA (US)

Correspondence Address: MERCHANT & GOULD PC, P.O. BOX 2903, MINNEAPOLIS, MN, 55402-0903, US

(73) Assignee: GENENTECH, INC., South San Francisco, CA (US)

(21) Appl. No.: 10/485,489

(22) PCT Filed: Jul. 24, 2002
(86) PCT No.: PCT/US02/23487

Related U.S. Application Data
(60) Provisional application No. 60/310,114, filed on Aug. 3, 2001.
(60) Provisional application No. 60/377,171, filed on Apr. 30, 2002.

(51) Int. Cl. C07K 14/705; C07H 21/04; C12N 1/18; C12N 5/06; C12N 15/74

(52) U.S. Cl. 530/350; 536/23.5; 435/69.1; 435/252.33; 435/254.2; 435/358; 435/320.1; 435/6

(57) ABSTRACT

Novel, receptors, referred to herein as “TACIs” and “BR3”, agonists and antagonists thereof, and method of using TACIs and BR3, as well as agonists and antagonists thereof, to modulate, for example, activity of tumor necrosis factor (TNF) and TNFR-related molecules, including members of the TNF and TNFR families referred to as TALL-1, APRIL, TACI, and BCMA, are provided. Methods for in vitro, in situ, and/or in vivo diagnosis and/or treatment of mammalian cells or pathological conditions associated with such TNF and TNFR-related molecules are further provided.

### FIELD OF THE INVENTION

This invention relates generally to novel receptors, referred to herein as “TACIs” and “BR3”, to agonists and antagonists thereof, and to methods of using TACIs and BR3, as well as agonists or antagonists thereof, to modulate for example, activity of tumor necrosis factor (TNF) and TNFR-related molecules, including members of the TNF and TNFR families referred to as TALL-1, APRIL, TACI, and BCMA. The invention also relates to methods for in vitro, in situ, and/or in vivo diagnosis and/or treatment of mammalian cells or pathological conditions associated with such TNF and TNFR-related molecules.

### BACKGROUND OF THE INVENTION

Various molecules, such as tumor necrosis factor-α(“TNF-α”), tumor necrosis factor-β (“TNF-α” or “lymphotoxin-α”), lymphotoxin-β (“LT-β”), CD30 ligand, CD27 ligand, CD40 ligand, OX-40 ligand, 4-1BB ligand, Apo-1 ligand (also referred to as Fas ligand or CD95 ligand), Apo-2 ligand (also referred to as TRAIL), Apo-3 ligand (also referred to as TWEAK), APRIL, OPG ligand (also referred to as RANK ligand, ODF, or TRANCE), and TALL-1 (also referred to as BlyS, BAFF or THANK) have been identified as members of the tumor necrosis factor (“TNF”) family of cytokines [See, e.g., Gruss and Dower, Blood, 85:3378-3404 (1995); Schmid et al., Proc. Natl. Acad. Sci., 83:1881 (1986); Dealtry et al., Eur. J. Immunol., 17:689 (1987); Pitti et al., J. Biol. Chem., 271:12687-12690 (1996); Wiley et al., Immunity, 3:673-682 (1995); Browning et al., Cell, 72:847-856 (1993); Armitage et al. Nature, 357:80-82 (1992), WO 97/01633 published Jan. 16, 1997; WO 97/25428 published Jul. 17, 1997; Marsters et al., Curr. Biol., 8:525-528 (1998); Chicheportiche et al., Biol. Chem., 272:32401-32410 (1997); Hahne et al., J. Exp. Med., 188:1185-1190 (1998); WO98/28426 published Jul. 2, 1998; WO98/46751 published Oct. 22, 1998; WO/98/18921 published May 7, 1998; Moore et al., Science, 285:260-263 (1999); Shu et al., J. Leukocyte Biol., 65:680 (1999); Schneider et al., J. Exp. Med., 189:1747-1756 (1999); Mukhopadhyay et al., J. Biol. Chem., 274:15978-15981 (1999)]. Among these molecules, TNF-α, TNF-β, CD30 ligand, 4-1BB ligand, Apo-1 ligand, Apo-2 ligand (Apo2L/TRAIL) and Apo-3 ligand (TWEAK) have been reported to be involved in apoptotic cell death.

Various molecules in the TNF family also have purported role(s) in the function or development of the immune system [Gruss et al., Blood, 85:3378 (1995)]. Zheng et al. have reported that TNF-α is involved in post-stimulation apoptosis of CD8-positive T cells [Zheng et al., Nature, 377:348-351 (1995)]. Other investigators have reported that CD30 ligand may be involved in deletion of self-reactive T cells in the thymus [Amakawa et al., Cold Spring Harbor Laboratory Symposium on Programmed Cell Death, Abstr. No. 10, (1995)]. CD40 ligand activates many functions of B cells, including proliferation, immunoglobulin secretion, and survival [Renshaw et al., J. Exp. Med., 180:1889 (1994)]. Another recently identified TNF family cytokine, TALL-1 (BlyS), has been reported, under certain conditions, to induce B cell proliferation and immunoglobulin secretion. [Moore et al., supra; Schneider et al., supra; Mackay et al., J. Exp. Med., 190:1697 (1999); Shu et al., J. Leukocyte Biol., 65:680-683 (1999); Gross et al., Nature, 404:995-999 (2000)].

Mutations in the mouse Fas/Apo-1 receptor or ligand genes (called lpr and gld, respectively) have been associated with some autoimmune disorders, indicating that Apo-1 ligand may play a role in regulating the clonal deletion of self-reactive lymphocytes in the periphery [Krammer et al., Curr. Op. Immunol., 6:279-289 (1994); Nagata et al., Science, 267:1449-1456 (1995)]. Apo-1 ligand is also reported to induce post-stimulation apoptosis in CD4-positive T lymphocytes and in B lymphocytes, and may be involved in the elimination of activated lymphocytes when their function is no longer needed [Krammer et al., supra; Nagata et al., supra]. Agonist mouse monoclonal antibodies specifically binding to the Apo-1 receptor have been reported to exhibit cell killing activity that is comparable to or similar to that of TNF-α [Yonehara et al., J. Exp. Med., 169:1747-1756 (1989)].

The TNF-related ligand called OPG ligand (also referred to as RANK ligand, TRANCE, or ODF) has been reported in the literature to have some involvement in certain immunoregulatory activities. WO98/28426 published Jul. 2, 1998 describes the ligand (referred to therein as RANK ligand) as a Type 2 transmembrane protein, which in a soluble form, was found to induce maturation of dendritic cells, enhance CD1a+ dendritic cell allo-stimulatory capacity in a MLR, and enhance the number of viable human peripheral blood T cells in vitro in the presence of TGF-beta. [see also, Anderson et al., Nature, 390:175-179 (1997)]. The WO98/28426 reference also discloses that the ligand enhanced production of TNF-alpha by one macrophage tumor cell line (called RAW264.7; ATCC TIB71), but did not stimulate nitric oxide production by those tumor cells.

The putative roles of OPG ligand/TRANCE/ODF in modulating dendritic cell activity [see, e.g., Wong et al., J. Exp. Med., 186:2075-2080 (1997); Wong et al:, J. Leukocyte Biol., 65:715-724 (1999); Josien et al., J. Immunol., 162:2562-2568 (1999); Josien et al., J. Exp. Med., 191495-501 (2000)] and in influencing T cell activation in an immune response [see, e.g., Bachmann et al., J. Exp. Med., 189:1025-1031 (1999); Green et al., J. Exp. Med., 189:1017-1020 (1999)] have been explored in the literature. Kong et al., Nature, 397:315-323 (1999) report that mice with a disrupted opgl gene showed severe osteoporosis, lacked osteoclasts, and exhibited defects in early differentiation of T and B lymphocytes. Kong et al. have further reported that systemic activation of T cells in vivo led to an OPGL-mediated increase in osteoclastogenesis and bone loss. [Kong et al., Nature, 402:304-308 (1999)].

Induction of various cellular responses mediated by such TNF family cytokines is believed to be initiated by their binding to specific cell receptors. Previously, two distinct TNF receptors of approximately 55-kDa (TNFR1) and 75-kDa (TNFR2) were identified [Hohman et al., J. Biol. Chem., 264:14927-14934 (1989); Brockhaus et al., Proc. Natl. Acad. Sci., 87:3127-3131 (1990); EP 417,563, published Mar. 20, 1991; Loetscher et al., Cell, 61:351 (1990); Schall et al., Cell, 61:361 (1990); Smith et al., Science, 248:1019-1023 (1990); Lewis et al., Proc. Natl. Acad. Sci., 88:2830-2834 (1991); Goodwin et al., Mol. Cell. Biol., 11:3020-3026 (1991)]. Those TNFRs were found to share the typical structure of cell surface receptors including extracellular, transmembrane and intracellular regions. The extracellular portions of both receptors were found naturally also as soluble TNF-binding proteins [Nophar, Y. et al., EMBO J., 9:3269 (1990); and Kohno, T. et al., Proc. Natl. Acad. Sci. U.S.A., 87:8331 (1990); Hale et al., J. Cell. Biochem. Supplement 15F, 1991, p. 113 (P424)].

The extracellular portion of type 1 and type 2 TNFRs (TNFR1 and TNFR2) contains a repetitive amino acid sequence pattern of four cysteine-rich domains (CRDs) designated 1 through 4, starting from the NH₂-terminus. [Schall et al., supra; Loetscher et al., supra; Smith et al., supra; Nophar et al., supra; Kohno et al., supra; Banner et al., Cell, 73:431-435 (1993)]. A similar repetitive pattern of CRDs exists in several other cell-surface proteins, including the p75 nerve growth factor receptor (NGFR) [Johnson et al., Cell, 47:545 (1986); Radeke et al., Nature, 325:593 (1987)], the B cell antigen CD40 [Stamenkovic et al., EMBO J., 8:1403 (1989)], the T cell antigen OX40 [Mallet et al., EMBO J., 9:1063 (1990)] and the Fas antigen [Yonehara et al., supra and Itoh et al., Cell, 66:233-243 (1991)]. CRDs are also found in the soluble TNFR (sTNFR)-like T2 proteins of the Shope and myxoma poxviruses [Upton et al., Virology, 160:20-29 (1987); Smith et al., Biochem. Biophys. Res. Commun., 176:335 (1991); Upton et al., Virology, 184:370 (1991)]. Optimal alignment of these sequences indicates that the positions of the cysteine residues are well conserved. These receptors are sometimes collectively referred to as members of the TNF/NGF receptor superfamily.

The TNF family ligands identified to date, with the exception of lymphotoxin-α, are typically type II transmembrane proteins, whose C-terminus is extracellular. In contrast, most receptors in the TNF receptor (TNFR) family identified to date are typically type I transmembrane proteins. In both the TNF ligand and receptor families, however, homology identified between family members has been found mainly in the extracellular domain (“ECD”). Several of the TNF family cytokines, including TNF-α, Apo-1 ligand and CD40 ligand, are cleaved proteolytically at the cell surface; the resulting protein in each case typically forms a homotrimeric molecule that functions as a soluble cytokine. TNF receptor family proteins are also usually cleaved proteolytically to release soluble receptor ECDs that can function as inhibitors of the cognate cytokines.

The TNFR family member, referred to as RANK, has been identified as a receptor for OPG ligand (see WO98/28426 published Jul. 2, 1998; Anderson et al., Nature, 390:175-179 (1997); Lacey et al., Cell, 93:165-176 (1998). Another TNFR-related molecule, called OPG (FDCR-1 or OCIF), has also been identified as a receptor for OPG ligand. [Simonet et al., Cell, 89:309 (1997); Yasuda et al., Endocrinology, 139:1329 (1998); Yun et al., J. Immunol., 161:6113-6121 (1998)]. Yun et al., supra, disclose that OPG/FDCR-1/OCIF is expressed in both a membrane-bound form and a secreted form and has a restricted expression pattern in cells of the immune system, including dendritic cells, EBV-transformed B cell lines and tonsillar B cells. Yun et al. also disclose that in B cells and dendritic cells, expression of OPG/FDCR-1/OCIF can be up-regulated by CD40, a molecule involved in B cell activation. However, Yun et al. acknowledge that how OPG/FDCR-1/OCIF functions in the regulation of the immune response is unknown.

More recently, other members of the TNFR family have been identified. In von Bulow et al., Science, 278:138-141 (1997), investigators describe a plasma membrane receptor referred to as Transmembrane Activator and CAML-Interactor or “TACI”. The TACI receptor is reported to contain a cysteine-rich motif characteristic of the TNFR family. In an in vitro assay, cross linking of TACI on the surface of transfected Jurkat cells with TACI-specific antibodies led to activation of NF-KB. [see also, WO 98/39361 published Sep. 18, 1998]. TACI knockout mice have been reported to have hyperresponsive B cells, while BCMA null mice had no discernable phenotype [Yan et al., Nature Immunology, 2:638-643 (2001); von Bulow et al., Immunity, 14:573-582 (2001); Xu et al., Mol. Cell. Biology, 21:4067-4074 (2001)].

Laabi et al., EMBO J., 11:3897-3904 (1992) reported identifying a new gene called “BCM” whose expression was found to coincide with B cell terminal maturation. The open reading frame of the BCM normal cDNA predicted a 184 amino acid long polypeptide with a single transmembrane domain. These investigators later termed this gene “BCMA.” [Laabi et al., Nucleic Acids Res., 22:1147-1154 (1994)]. BCMA mRNA expression was reported to be absent in human malignant B cell lines which represent the pro-B lymphocyte stage, and thus, is believed to be linked to the stage of differentiation of lymphocytes [Gras et al., Int. Immunology, 7:1093-1106 (1995)]. In Madry et al., Int. Immunology, 10:1693-1702 (1998), the cloning of murine BCMA cDNA was described. The murine BCMA cDNA is reported to encode a 185 amino acid long polypeptide having 62% identity to the human BCMA polypeptide. Alignment of the murine and human BCMA protein sequences revealed a conserved motif of six cysteines in the N-terminal region, suggesting that the BCMA protein belongs to the TNFR superfamily [Madry et al., supra].

The Tall-1 (BlyS) ligand has been reported to bind the TACI and BCMA receptors [Gross et al., supra, (2000); Thompson et al., J. Exp. Med., 192:129-135 (2000); Yan et al., supra, (2000); Marsters et al., Curr. Biol., 10:785-758 (2000); WO 00/40716 published Jul. 13, 2000; WO 00/67034 published Nov. 9, 2000]. TACI and BCMA have likewise been reported to bind to the ligand known as April.

In Marsters et al., Curr. Biol., 6:750 (1996), investigators describe a full length native sequence human polypeptide, called Apo-3, which exhibits similarity to the TNFR family in its extracellular cysteine-rich repeats and resembles TNFR1 and CD95 in that it contains a cytoplasmic death domain sequence [see also Marsters et al., Curr. Biol., 6:1669 (1996)]. Apo-3 has also been referred to by other investigators as DR3, wsl-1, TRAMP, and LARD [Chinnaiyan et al., Science, 274:990 (1996); Kitson et al., Nature, 384:372 (1996); Bodmer et al., Immunity, 6:79 (1997); Screaton et al., Proc. Natl. Acad. Sci., 94:4615-4619 (1997)].

Pan et al. have disclosed another TNF receptor family member referred to as “DR4” (Pan et al., Science, 276:111-113 (1997); see also WO98/32856 published Jul. 30, 1998]. The DR4 was reported to contain a cytoplasmic death domain capable of engaging the cell suicide apparatus. Pan et al. disclose that DR4 is believed to be a receptor for the ligand known as Apo2L/TRAIL.

In Sheridan et al., Science, 277:818-821 (1997) and Pan et al., Science, 277:815-818 (1997), another molecule believed to be a receptor for Apo2L/TRAIL is described [see also, WO98/51793 published Nov. 19, 1998; WO98/41629 published Sep. 24, 1998]. That molecule is referred to as DR5 (it has also been alternatively referred to as Apo-2; TRAIL-R, TR6, Tango-63, hAPO8, TRICK2 or KILLER [Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999]. Like DR4, DR5 is reported to contain a cytoplasmic death domain and be capable of signaling apoptosis. The crystal structure of the complex formed between Apo-2L/TRAIL and DR5 is described in Hymowitz et al., Molecular Cell, 4:563-571 (1999).

Yet another death domain-containing receptor, DR6, was recently identified [Pan et al., FEBS Letters, 431:351-356 (1998)]. Aside from containing four putative extracellular cysteine rich domains and a cytoplasmic death domain, DR6 is believed to contain a putative leucine-zipper sequence that overlaps with a proline-rich motif in the cytoplasmic region. The proline-rich motif resembles sequences that bind to src-homology-3 domains, which are found in many intracellular signal-transducing molecules. In contrast to other death domain-containing receptors referred to above, DR6 does not induce cell death in the apoptosis sensitive indicator cell line, MCF-7, suggesting an alternate function for this receptor. Consistent with this observation, DR6 is presently believed not to associate with death-domain containing adapter molecules, such as FADD, RAIDD and RIP, that mediate downstream signaling from activated death receptors [Pan et al., FEBS Lett., 431:351 (1998)].

A further group of recently identified receptors are referred to as “decoy receptors,” which are believed to function as inhibitors, rather than transducers of signaling. This group includes DCR1 (also referred to as TRID, LIT or TRAIL-R3) [Pan et al., Science, 276:111-113 (1997); Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998)] and DCR2 (also called TRUNDD or TRAIL-R4) [Marsters et al., Curr. Biol., 7:1003-1006 (1997); Pan et al., FEBS Letters, 424:41-45 (1998); Degli-Esposti et al., Immunity, 7:813-820 (1997)], both cell surface molecules, as well as OPG [Simonet et al., supra; Emery et al., infra] and DCR3 [Pitti et al., Nature, 396:699-703 (1998)], both of which are secreted, soluble proteins.

Additional newly identified members of the TNFR family include CAR1, HVEM, GITR, ZTNFR-5, NTR-1, and TNFL1 [Brojatsch et al., Cell, 87:845-855 (1996); Montgomery et al., Cell, 87:427-436 (1996); Marsters et al., J. Biol. Chem., 272:14029-14032 (1997); Nocentini et al., Proc. Natl. Acad. Sci. USA 94:6216-6221 (1997); Emery et al., J. Biol. Chem., 273:14363-14367 (1998); WO99/04001 published Jan. 28, 1999; WO99/07738 published Feb. 18, 1999; WO99/33980 published Jul. 8, 1999].

As reviewed recently by Tewari et al., TNFR1, TNFR2 and CD40 modulate the expression of proinflammatory and costimulatory cytokines, cytokine receptors, and cell adhesion molecules through activation of the transcription factor, NF-κB [Tewari et al., Curr. Op. Genet. Develop., 6:39-44 (1996)]. NF-κB is the prototype of a family of dimeric transcription factors whose subunits contain conserved Rel regions [Verma et al., Genes Develop., 9:2723-2735 (1996); Baldwin, Ann. Rev. Immunol., 14:649-681 (1996)]. In its latent form, NF-κB is complexed with members of the IκB inhibitor family; upon inactivation of the IκB in response to certain stimuli, released NF-κB translocates to the nucleus where it binds to specific DNA sequences and activates gene transcription. As described above, the TNFR members identified to date either include or lack an intracellular death domain region. Some TNFR molecules lacking a death domain, such as TNFR2, CD40, HVEM, and GITR, are capable of modulating NF-κB activity. [see, e.g., Lotz et al., J. Leukocyte Biol., 60:1-7 (1996)].

For a review of the TNF family of cytokines and their receptors, see Ashkenazi and Dixit, Science, 281:1305-1308 (1998); Golstein, Curr. Biol., 7:750-753 (1997); Gruss and Dower, supra, and Nagata, Cell, 88:355-365 (1997).

### SUMMARY OF THE INVENTION

Applicants have identified novel molecules referred to as “TACIs” and “BR3”. TACIs polypeptide has been characterized as having a single cysteine-rich domain, in contrast to the full-length human TACI molecule described in von Bulow et al., supra, which includes two cysteine-rich domains. Likewise, BR3 polypeptide as described herein has been characterized as having a single cysteine-rich domain. Applicants have surprisingly found that the TNF family ligands referred to as TALL-1 and April bind to the TACIs receptor. Applicants have also surprisingly found that the TNF family ligand referred to as TALL-1 binds to BR3 receptor. In contrast to the TACI and BCMA receptors, BR3 does not appear to bind the ligand, April, and does not activate the NF-KB pathway. The present invention thus provides for novel methods of using antagonists or agonists of these TNF-related ligands and receptors. The antagonists and agonists described herein find utility for, among other things, in vitro, in situ, or in vivo diagnosis or treatment of mammalian cells or pathological conditions associated with the presence (or absence) of TALL-1, APRIL, TACI, BCMA, TACIs, or BR3.

In one embodiment, the invention provides isolated nucleic acid molecules comprising DNA encoding a TACIs polypeptide. In certain aspects, the isolated nucleic acid comprises DNA encoding the TACIs polypeptide having amino acid residues 1 to 246 or 1 to 119 of FIG. 5B (SEQ ID NO:14), or is complementary to such encoding nucleic acid sequences, and remains stably bound to it under at least moderate, and optionally, under high stringency conditions.

In another embodiment, the invention provides vectors comprising DNA encoding a TACIs polypeptide. A host cell comprising such a vector is also provided. By way of example, the host cells may be CHO cells, E. coli, or yeast. A process for producing TACIs polypeptides is further provided and comprises culturing host cells under conditions suitable for expression of TACIs polypeptide and recovering TACIs polypeptide from the cell culture.

In another embodiment, the invention provides isolated TACIs polypeptides. In particular, the invention provides isolated TACIs polypeptides which include an amino acid sequence comprising residues 1 to 246 of FIG. 5B (SEQ ID NO:14). Additional embodiments of the present invention are directed to isolated extracellular domain sequences of TACIs polypeptide comprising amino acids 1 to 119 of the amino acid sequence shown in FIG. 5B (SEQ ID NO:14), or fragments thereof, particularly biologically active fragments.

In another embodiment, the invention provides chimeric molecules comprising TACIs polypeptide or extracellular domain sequence or other fragment thereof fused to a heterologous polypeptide or amino acid sequence. An example of such a chimeric molecule comprises a TACIs polypeptide fused to an epitope tag sequence or a Fc region of an immunoglobulin.

In another embodiment, the invention provides an antibody which specifically binds to a TACIs polypeptide or extracellular domain thereof. Optionally, the antibody is a monoclonal antibody.

In a still further embodiment, the invention provides diagnostic and therapeutic methods using TACIs polypeptide or DNA encoding TACIs polypeptide.

In another embodiment, the invention provides isolated nucleic acid molecules comprising DNA encoding a BR3 polypeptide. In certain aspects, the isolated nucleic acid comprises DNA encoding the BR3 polypeptide having amino acid residues 1 to 184, 1 to 77 or 2 to 62 of FIG. 6B (SEQ ID NO:16), or is complementary to such encoding nucleic acid sequences, and remains stably bound to it under at least moderate, and optionally, under high stringency conditions.

In another embodiment, the invention provides vectors comprising DNA encoding a BR3 polypeptide. A host cell comprising such a vector is also provided. By way of example, the host cells may be CHO cells, E. coli, or yeast. A process for producing BR3 polypeptides is further provided and comprises culturing host cells under conditions suitable for expression of BR3 polypeptide and recovering BR3 polypeptide from the cell culture.

In another embodiment, the invention provides isolated BR3 polypeptides. In particular, the invention provides isolated BR3 polypeptides which include an amino acid sequence comprising residues 1 to 184, 1 to 77 or 2 to 62 of FIG. 6B (SEQ ID NO:16). Additional embodiments of the present invention are directed to isolated extracellular domain sequences of BR3 polypeptide comprising amino acids 1 to 77 or 2 to 62 of the amino acid sequence shown in FIG. 6B (SEQ ID NO:16), or fragments thereof.

In another embodiment, the invention provides chimeric molecules comprising a BR3 polypeptide or extracellular domain sequence or other fragment thereof fused to a heterologous polypeptide or amino acid sequence. An example of such a chimeric molecule comprises a BR3 polypeptide fused to an epitope tag sequence or a Fc region of an immunoglobulin.

In another embodiment, the invention provides an antibody which specifically binds to a BR3 polypeptide or extracellular domain thereof. Optionally, the antibody is a monoclonal antibody.

In a still further embodiment, the invention provides diagnostic and therapeutic methods using BR3 polypeptide or DNA encoding BR3 polypeptide.

The methods of the invention include methods to treat pathological conditions or diseases in mammals associated with or resulting from increased or enhanced TALL-1 or APRIL expression and/or activity. In the methods of treatment, TALL-1 antagonists or APRIL antagonists may be administered to the mammal suffering from such pathological condition or disease. The TALL-1 antagonists and APRIL antagonists contemplated for use in the invention include TACIs receptor immunoadhesins or BR3 receptor immunoadhesins, as well as antibodies against the TACIs receptor or BR3 receptor, which preferably block or reduce the respective receptor binding or activation by TALL-1 ligand and/or APRIL ligand. For instance, TACIs receptor immunoadhesins may be employed to treat rheumatoid arthritis or multiple sclerosis. The TALL-1 antagonists and APRIL antagonists contemplated for use further include anti-TALL-1 antibodies or anti-APRIL antibodies which are capable of blocking or reducing binding of the respective ligands to the TACIs or BR3 receptors. Still further antagonist molecules include covalently modified forms, or fusion proteins, comprising TACIs or BR3. By way of example, such antagonists may include pegylated TACIs or BR3 and TACIs or BR3 fused to heterologous sequences such as epitope tags or leucine zippers. Optionally, the antagonist molecule(s) employed in the methods will be capable of blocking or neutralizing the activity of both TALL-1 and APRIL, e.g., a dual antagonist which blocks or neutralizes activity of both TALL-1 and APRIL. Optionally, the antagonist molecule(s) employed in the methods will be capable of blocking or neutralizing the activity of TALL-1 but not APRIL, e.g., an antagonist (such as a BR3 immunoadhesin) which blocks or neutralizes activity of TALL-1. For instance, a BR3 immunoadhesin may be employed to treat an autoimmune disorder such as lupus. The methods contemplate the use of a single type of antagonist molecule or a combination of two or more types of antagonist.

In another embodiment of the invention, there are provided methods for the use of TALL-1 antagonists to block or neutralize the interaction between TALL-1 and TACIs and/or BR3. Such antagonists may also block or neutralize the interaction between TALL-1 and TACI and/or BCMA. For example, the invention provides a method comprising exposing a mammalian cell, such as a white blood cell (preferably a B cell), to one or more TALL-1 antagonists in an amount effective to decrease, neutralize or block activity of the TALL-1 ligand. The cell may be in cell culture or in a mammal, e.g. a mammal suffering from, for instance, an immune related disease or cancer. Thus, the invention includes a method for treating a mammal suffering from a pathological condition such as an immune related disease or cancer comprising administering an effective amount of one or more TALL-1 antagonists, as disclosed herein. In particular embodiments, the immune related disorder is an autoimmune disease such as arthritis or lupus.

The invention also provides methods for the use of APRIL antagonists to block or neutralize the interaction between APRIL and TACIs. Such antagonists may also block or neutralize the interaction between APRIL and TACI and/or BCMA. For example, the invention provides a method comprising exposing a mammalian cell, such as a white blood cell (preferably a B cell), to one or more APRIL antagonists in an amount effective to decrease, neutralize or block activity of the APRIL ligand. The cell may be in cell culture or in a mammal, e.g. a mammal suffering from, for instance, an immune related disease or cancer. Thus, the invention includes a method for treating a mammal suffering from a pathological condition such as an immune related disease or cancer comprising administering an effective amount of one or more APRIL antagonists, as disclosed herein.

The invention also provides compositions which comprise one or more TALL-1 antagonists or APRIL antagonists. Optionally, the compositions of the invention will include pharmaceutically acceptable carriers or diluents. Preferably, the compositions will include one or more TALL-1 antagonists or APRIL antagonists in an amount which is therapeutically effective to treat a pathological condition or disease.

The invention also provides articles of manufacture and kits which include one or more TALL-1 antagonists or APRIL antagonists.

In addition, the invention provides methods of using TACIs agonists or BR3 agonists to, for instance, stimulate or activate TACIs receptor or BR3 receptor. Such methods will be useful in treating pathological conditions characterized by or associated with insufficient TALL-1 or APRIL expression or activity such as immunodeficiency or cancer (such as by boosting the immune anti-cancer response). The TACIs agonists or BR3 agonists may comprise agonistic anti-TACIs or anti-BR3 antibodies. The agonistic activity of such TACIs agonists or BR3 agonists may comprise enhancing the activity of a native ligand for TACIs or BR3 or activity which is the same as or substantially the same as (i.e., mimics) the activity of a native ligand for TACIs or BR3.

Thus, the invention also provides compositions which comprise one or more TACIs agonists or BR3 agonists. Optionally, the compositions of the invention will include pharmaceutically acceptable carriers or diluents. Preferably, the compositions will include one or more TACIs agonists or BR3 agonists in an amount which is therapeutically effective to stimulate signal transduction by TACIs or BR3.

Further, the invention provides articles of manufacture and kits which include one or more TACIs agonists or BR3 agonists.

The invention also provides methods of conducting screening assays to identify candidate molecules, such as small molecule compounds, polypeptides or antibodies, which act as agonists or antagonists with respect to the interaction between TALL-1 and TACIs or BR3, or to the interaction between APRIL and TACIs.

### BRIEF DESCRIPTION OF THE DRAWINGS

FIGS. 1A-1B show a polynucleotide sequence encoding a native sequence human TACI (SEQ ID NO:1) (reverse complimentary sequence is provided in SEQ ID NO:2) and its putative amino acid sequence (SEQ ID NO:3).

FIG. 2 shows a polynucleotide sequence encoding a native sequence human BCMA (SEQ ID NO:4) (reverse complimentary sequence is provided in SEQ ID NO:5) and its putative amino acid sequence (SEQ ID NO:6).

FIG. 3 shows a polynucleotide sequence encoding a native sequence human TALL-1 (SEQ ID NO:7) (reverse complimentary sequence is provided in SEQ ID NO:8) and its putative amino acid sequence (SEQ ID NO:9).

FIGS. 4A-4B show a polynucleotide sequence encoding a native sequence human APRIL (SEQ ID NO:10) (reverse complimentary sequence is provided in SEQ ID NO:11) and its putative amino acid sequence (SEQ ID NO:12).

FIG. 5A shows a polynucleotide sequence (start and stop codons are underlined) encoding a native sequence human TACIs (SEQ ID NO:13) and FIG. 5B shows its putative amino acid sequence (SEQ ID NO:14).

FIG. 6A shows a polynucleotide sequence (start and stop codons are underlined) encoding a native sequence human BR3 (SEQ ID NO:15), and FIG. 6B shows its putative amino acid sequence (SEQ ID NO:16); FIG. 6C shows a polynucleotide sequence (start and stop codons are underlined) encoding murine BR3 (SEQ ID NO:17), and FIG. 9A shows its putative amino acid sequence (SEQ ID NO:18).

FIGS. 7A-7B show exemplary methods for calculating the % amino acid sequence identity of the amino acid sequence designated “Comparison Protein” to the amino acid sequence designated “PRO”. For purposes herein, the “PRO” sequence may be the TACI, BCMA, TALL-1, APRIL, TACIs, or BR3 sequences referred to in the Figures herein.

FIG. 8 shows an alignment of two amino acid sequences for the TACI receptor, referred to as “hTACI (265)” (SEQ ID NO:19), believed to be a spliced variant, and “hTACI”, also referred to in FIGS. 1A-1B (SEQ ID NO:3).

FIG. 9A shows a sequence alignment of human (SEQ IS NO:16) and murine BR3 (SEQ ID NO:18). Amino acids that are identical in human and murine BR3 are shown in bold. Conserved amino acids are indicated by a plus sign. The region containing four cysteine residues is underlined and the predicted membrane-spanning region is doubly underlined. FIG. 9B shows Northern Blot analysis of BR3. Human (left) and mouse (right) multiple tissue northern blots (Clontech) were probed with ³²P-labelled cDNA fragments corresponding to the coding region of human or murine BR3. FIG. 9C shows PCR analysis of human multiple tissue cDNA panel (Clontech). cDNA fragments were amplified using gene specific primers. Lanes 1-9: 1, PBL; 2, resting CD4+ cells; 3, activated CD4+ cells; 4, resting CD8+ cells; 5, activated CD8+ cells; 6, resting CD19+ cells; 7, activated CD 19+ cells; 8, lymph node; 9, spleen.

FIGS. 10A-10D shows the results of assays conducted and showing BR3 is a specific receptor for TALL-1 but not for APRIL and fails to activate the NF-KB pathway. (a) COS 7 transfected with hBR3 (1,2) or TACI (3,4) were incubated with conditioned medium containing AP-TALL-1 (1,3) or AP-April (2,4). Cells were washed, fixed, and stained for the AP activity in situ. (b) COS7 cells transfected with TALL-1 (1,2) or April (3,4) were incubated with hBR3-hFc (1,3) or TACI-hFc (2,4). Cells were washed, fixed, and bound receptor-hFc protein was detected using a biotinylated goat anti-human antibody followed by Cy3-streptavidin. (c) BR3-hFc (1,2) or TACI-hFc (3,4) was incubated with Flag-TALL-1 (1,3) or Flag-April (2,4). The receptor-Fc fusion proteins precipitated with protein-A-agarose were subjected to immunoblotting with anti-Flag antibody. Equivalent amounts of ligand (middle panel) or receptor-hFc (bottom) were used in the binding experiment. (d) 293E cells (Invitrogen) were transfected with 0.25 ug of a NF-kB luciferase reporter gene construct, 25 ng pRL-TK, and indicated amounts of expression constructs encoding hBR3, mBR3, TACI and BCMA. NF-kB activation was determined 20-24 hours later using the Dual-Luciferase reporter assay kit (Promega).

FIGS. 11A-11D illustrate the results of assays showing BR3-Fc is effective in treating lupus. FIGS. 11A-11B show BR3-Fc blocked proteinurea in NZB×NZW (F1) mice; FIG. 11C shows BR3-Fc treated animals exhibited enhanced survival; FIG. 11D shows BR3-Fc treated animals had decreased presence of anti-dsDNA antibodies.

### DETAILED DESCRIPTION OF THE INVENTION

I. Definitions

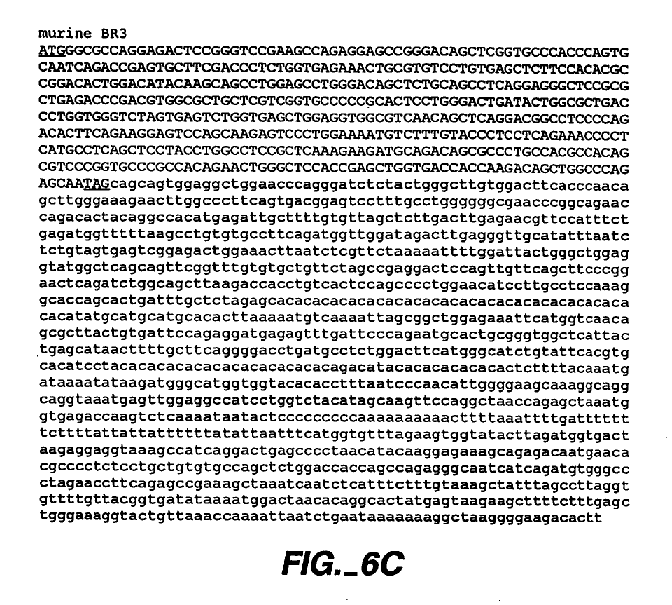

The terms “BR3”, “BR3 polypeptide” or “BR3 receptor” when used herein encompass “native sequence BR3 polypeptides” and “BR3 variants” (which are further defined herein). “BR3” is a designation given to those polypeptides which are encoded by the nucleic acid molecules comprising the polynucleotide sequences shown in FIG. 6 and variants or fragments thereof, nucleic acid molecules comprising the sequence shown in the FIG. 6 and variants thereof as well as fragments of the above. The BR3 polypeptides of the invention may be isolated from a variety of sources, such as from human tissue types or from another source, or prepared by recombinant and/or synthetic methods.

A “native sequence” BR3 polypeptide comprises a polypeptide having the same amino acid sequence as the corresponding BR3 polypeptide derived from nature. Such native sequence BR3 polypeptides can be isolated from nature or can be produced by recombinant and/or synthetic means. The term “native sequence BR3 polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The BR3 polypeptides of the invention include the BR3 polypeptide comprising or consisting of the contiguous sequence of amino acid residues 1 to 184 of FIG. 6B (SEQ ID NO:16).

A BR3 “extracellular domain” or “ECD” refers to a form of the BR3 polypeptide which is essentially free of the transmembrane and cytoplasmic domains. Ordinarily, a BR3 polypeptide ECD will have less than about 1% of such transmembrane and/or cytoplasmic domains and preferably, will have less than about 0.5% of such domains. It will be understood that any transmembrane domain(s) identified for the BR3 polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. ECD forms of BR3 include those comprising amino acids 1 to 77 or 2 to 62 of FIG. 6B.

“BR3 variant” means a BR3 polypeptide having at least about 80% amino acid sequence identity with the amino acid sequence of a native sequence full length BR3 or BR3 ECD. Optionally, the BR3 variant includes a single cysteine rich domain. Preferably such BR3 variant acts as an antagonist or agonist as defined below. Such BR3 variant polypeptides include, for instance, BR3 polypeptides wherein one or more amino acid residues are added, or deleted, at the N- and/or C-terminus, as well as within one or more internal domains, of the full-length amino acid sequence. Fragments of the BR3 ECD are also contemplated. Ordinarily, a BR3 variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a BR3 polypeptide encoded by a nucleic acid molecule shown in FIG. 6 or a specified fragment thereof. BR3 variant polypeptides do not -encompass the native BR3 polypeptide sequence; Ordinarily, BR3 variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

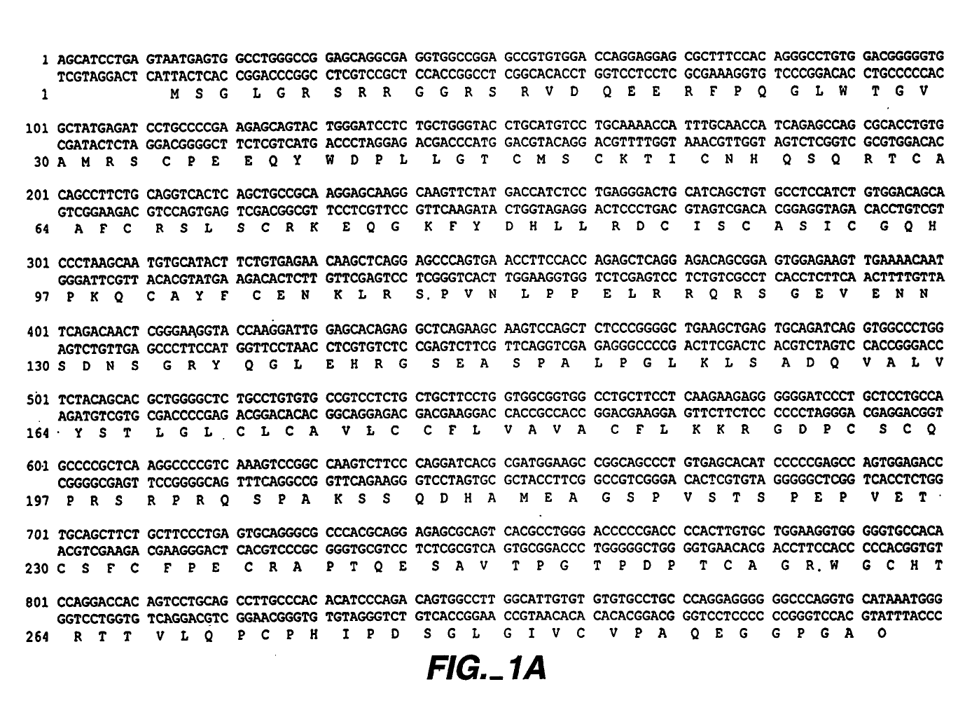

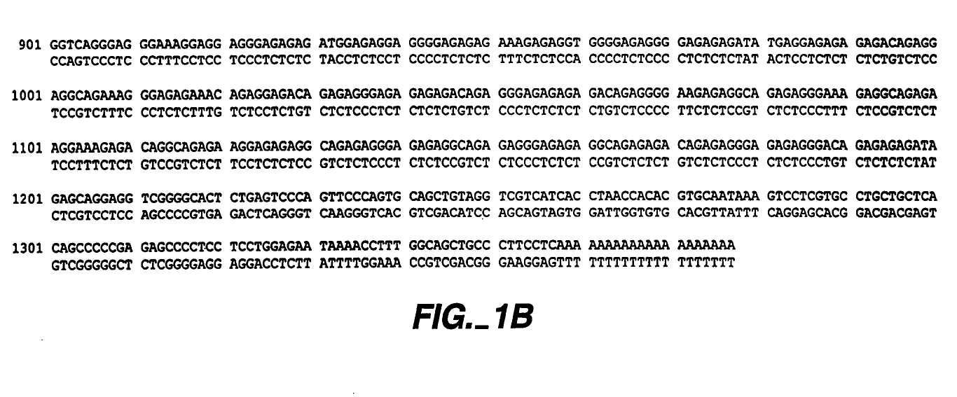

The terms “TACI” or “TACI polypeptide” or “TACI receptor” when used herein encompass “native sequence TACI polypeptides” and “TACI variants” (which are further defined herein). “TACI” is a designation given to those polypeptides which are encoded by the nucleic acid molecules comprising the polynucleotide sequences shown in FIG. 1 and variants or fragments thereof, nucleic acid molecules comprising the sequence shown in the FIG. 1 and variants thereof as well as fragments of the above. The TACI polypeptides of the invention may be isolated from a variety of sources, such as from human tissue types or from another source, or prepared by recombinant and/or synthetic methods.

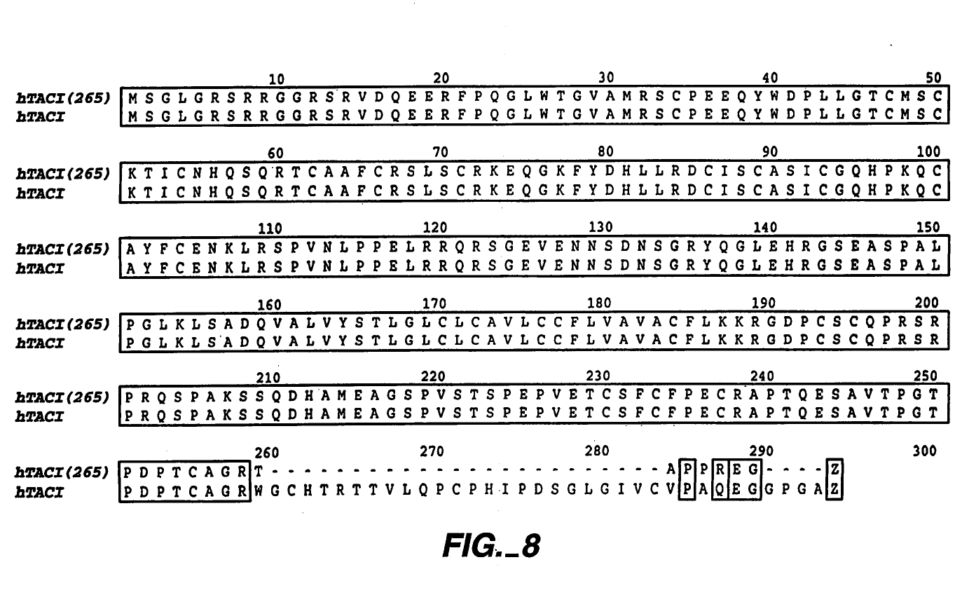

A “native sequence” TACI polypeptide comprises a polypeptide having the same amino acid sequence as the corresponding TACI polypeptide derived from nature. Such native sequence TACI polypeptides can be isolated from nature or can be produced by recombinant and/or synthetic means. The term “native sequence TACI polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The TACI polypeptides of the invention include but are not limited to the polypeptides described in von Bulow et al., supra and WO98/39361 published Sep. 11, 1998, the spliced variant (referred to as “hTACI(265)” above and shown in FIG. 8 (SEQ ID NO:19)), and the TACI polypeptide comprising the contiguous sequence of amino acid residues 1-293 of FIG. 1 (SEQ ID NO:3).

A TACI “extracellular domain” or “ECD” refers to a form of the TACI polypeptide which is essentially free of the transmembrane and cytoplasmic domains. Ordinarily, a TACI polypeptide ECD will have less than about 1% of such transmembrane and/or cytoplasmic domains and preferably, will have less than about 0.5% of such domains. It will be understood that any transmembrane domain(s) identified for the TACI polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. ECD forms of TACI include those described in von Bulow et al., supra and WO98/39361.

“TACI variant” means a TACI polypeptide having at least about 80% amino acid sequence identity with the amino acid sequence of a native sequence full length TACI or TACI ECD. Preferably such TACI variant acts as a TALL-1 antagonist or APRIL antagonist as defined below. Such TACI variant polypeptides include, for instance, TACI polypeptides wherein one or more amino acid residues are added, or deleted, at the N- and/or C-terminus, as well as within one or more internal domains, of the full-length amino acid sequence. Fragments of the TACI ECD are also contemplated. Ordinarily, a TACI variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a TACI polypeptide encoded by a nucleic acid molecule shown in FIG. 1 or a specified fragment thereof. TACI variant polypeptides do not encompass the native TACI polypeptide sequence. Ordinarily, TACI variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

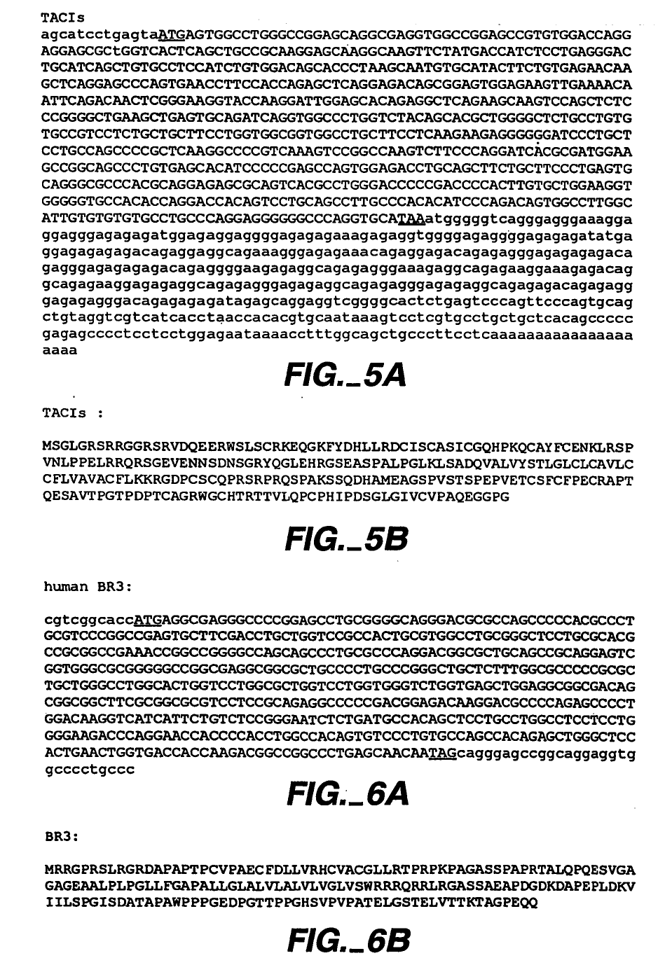

The term “TACIs” when used herein refers to polypeptides comprising the amino acid sequence of residues 1 to 246 of FIG. 5B, or fragments or variants thereof, and which comprise a single cysteine rich domain. Optionally, such TACIs polypeptides comprise the contiguous sequence of residues 1 to 246 of FIG. 5B. Optionally, such TACIs polypeptides are encoded by the nucleic acid molecules comprising the coding polynucleotide sequence shown in FIG. 5A. The TACIs polypeptides of the invention may be isolated from a variety of sources, such as from human tissue types or from another source, or prepared by recombinant and/or synthetic methods. The term “TACIs” expressly excludes those polypeptides defined herein as “TACI”. A “native sequence” TACIs polypeptide comprises a polypeptide derived from nature. Such native sequence TACIs polypeptides can be isolated from nature or can be produced by recombinant and/or synthetic means. A TACIs polypeptide may comprise a fragment or variant of the polypeptide shown in FIG. 5B and having at least about 80% amino acid sequence identity with the sequence shown in FIG. 5B, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a TACIs polypeptide encoded by an encoding nucleic acid sequence shown in FIG. 5A or a specified fragment thereof. Preferably such a TACIs variant acts as a TALL-1 antagonist or APRIL antagonist as defined below. Such variant polypeptides include, for instance, polypeptides wherein one or more amino acid residues are added, or deleted, at the N- and/or C-terminus, as well as within one or more internal domains, of the amino acid sequence shown in FIG. 5B.

A TACIs “extracellular domain” or “ECD” refers to a form of the TACIs polypeptide which is essentially free of the transmembrane and cytoplasmic domains. Ordinarily, a TACIs polypeptide ECD will have less than about 1% of such transmembrane and/or cytoplasmic domains and preferably, will have less than about 0.5% of such domains. It will be understood that any transmembrane domain(s) identified for the TACIs polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. ECD forms of TACIs include polypeptides comprising amino acid residues 1 to 119 of FIG. 5B, and optionally a sequence of contiguous amino acid residues 1 to 119 of FIG. 5B.

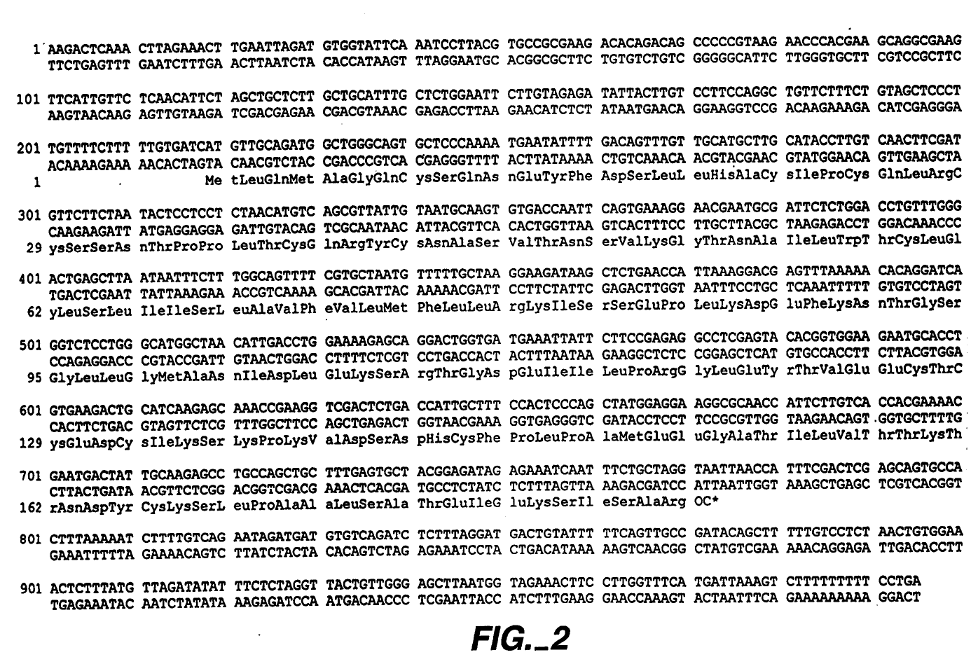

The terms “BCMA” or “BCMA polypeptide” or “BCMA receptor” when used herein encompass “native sequence BCMA polypeptides” and “BCMA variants” (which are further defined herein). “BCMA” is a designation given to those polypeptides which are encoded by the nucleic acid molecules comprising the polynucleotide sequences shown in FIG. 2 and variants thereof, nucleic acid molecules comprising the sequence shown in the FIG. 2 and variants thereof as well as fragments of the above. The BCMA polypeptides of the invention may be isolated from a variety of sources, such as from human tissue types or from another source, or prepared by recombinant and/or synthetic methods.

A “native sequence” BCMA polypeptide comprises a polypeptide having the same amino acid sequence as the corresponding BCMA polypeptide derived from nature. Such native sequence BCMA polypeptides can be isolated from nature or can be produced by recombinant and/or synthetic means. The term “native sequence BCMA polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The BCMA polypeptides of the invention include the polypeptides described in Laabi et al., EMBO J., 11:3897-3904 (1992); Laabi et al., Nucleic Acids Res., 22:1147-1154 (1994); Gras et al., Int. Immunology, 7:1093-1106 (1995); Madry et al., Int. Immunology, 10:1693-1702 (1998); and the BCMA polypeptide comprising the contiguous sequence of amino acid residues 1-184 of FIG. 2 (SEQ ID NO:6).

A BCMA “extracellular domain” or “ECD” refers to a form of the BCMA polypeptide which is essentially free of the transmembrane and cytoplasmic domains. Ordinarily, a BCMA polypeptide ECD will have less than about 1% of such transmembrane and/or cytoplasmic domains and preferably, will have less than about 0.5% of such domains. It will be understood that any transmembrane domain(s) identified for the BCMA polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. ECD forms of BCMA include those described in Laabi et al., EMBO J., 11:3897-3904 (1992); Laabi et al., Nucleic Acids Res., 22:1147-1154 (1994); Gras et al., Int. Immunology, 7:1093-1106 (1995); Madry et al., Int. Immunology, 10:1693-1702 (1998).

“BCMA variant” means a BCMA polypeptide having at least about 80% amino acid sequence identity with the amino acid sequence of a native sequence BCMA or BCMA ECD. Preferably such a BCMA variant acts as a TALL-1 antagonist or APRIL antagonist as defined below. Such BCMA variant polypeptides include, for instance, BCMA polypeptides wherein one or more amino acid residues are added, or deleted, at the N- and/or C-terminus, as well as within one or more internal domains, of the full-length amino acid sequence. Fragments of the BCMA ECD are also contemplated. Ordinarily, a BCMA variant polypeptide will have at least about 80% amino acid sequence identity,-more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a BCMA polypeptide encoded by a nucleic acid molecule shown in FIG. 2 or a specified fragment thereof. BCMA variant polypeptides do not encompass the native BCMA polypeptide sequence. Ordinarily, BCMA variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

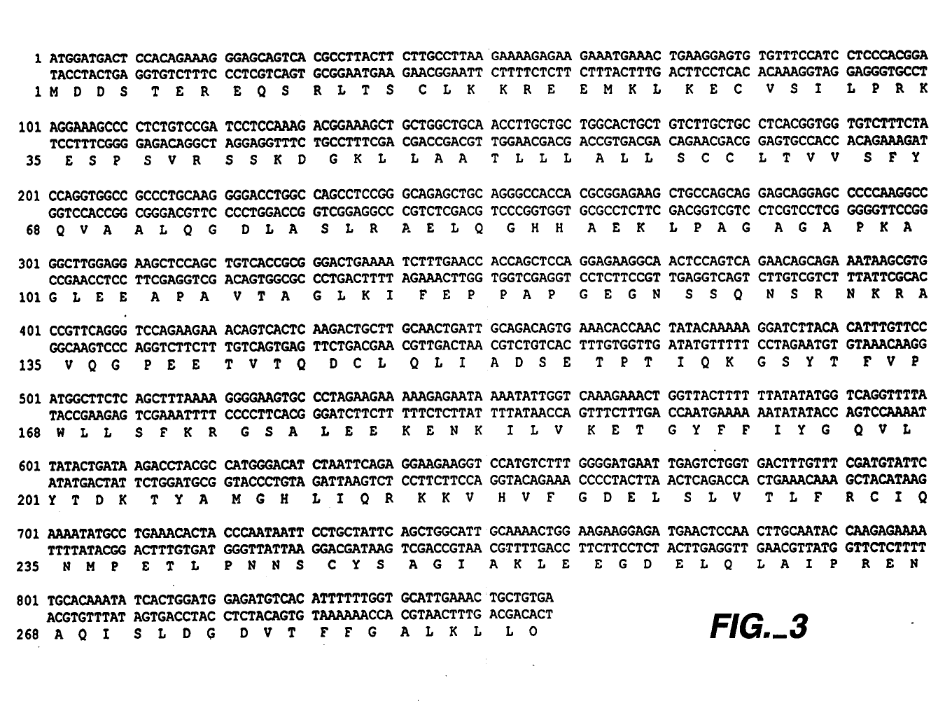

The terms “TALL-1” or “TALL-1 polypeptide” when used herein encompass “native sequence TALL-1 polypeptides” and “TALL-1 variants”. “TALL-1” is a designation given to those polypeptides which are encoded by the nucleic acid molecules comprising the polynucleotide sequences shown in FIG. 3 and variants thereof, nucleic acid molecules comprising the sequence shown in the FIG. 3, and variants thereof as well as fragments of the above which have the biological activity of the native sequence TALL-1. Variants of TALL-1 will preferably have at least 80%, more preferably, at least 90%, and even more preferably, at least 95% amino acid sequence identity with the native sequence TALL-1 polypeptide shown in FIG. 3. A “native sequence” TALL-1 polypeptide comprises a polypeptide having the same amino acid sequence as the corresponding TALL-1 polypeptide derived from nature. Such native sequence TALL-1 polypeptides can be isolated from nature or can be produced by recombinant and/or synthetic means. The term “native sequence TALL-1 polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “TALL-1” includes those polypeptides described in Shu et al., GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., supra; Schneider et al., supra; and Mukhopadhyay et al., supra.

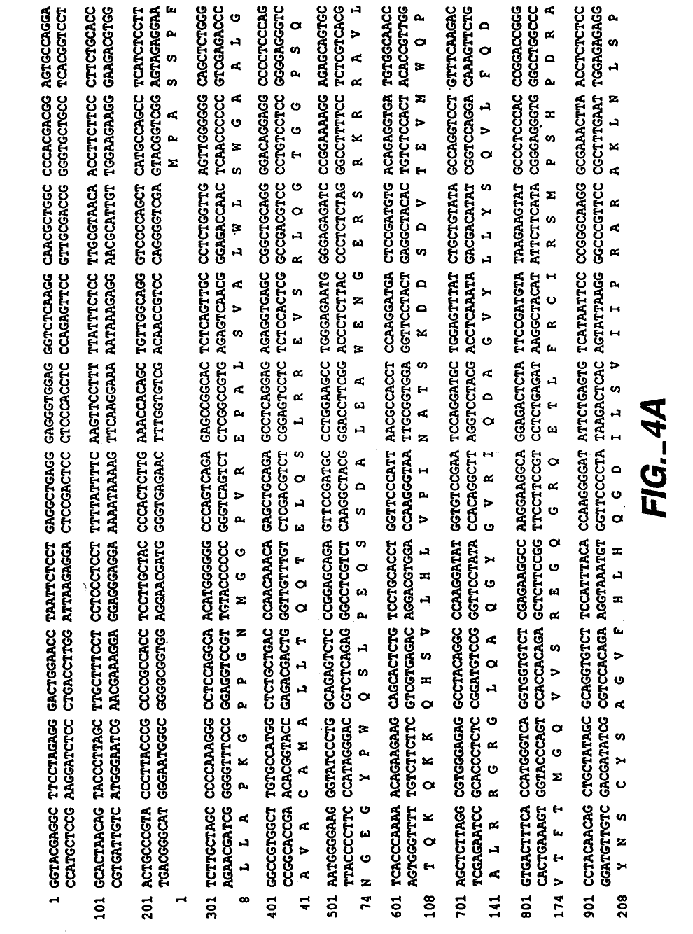

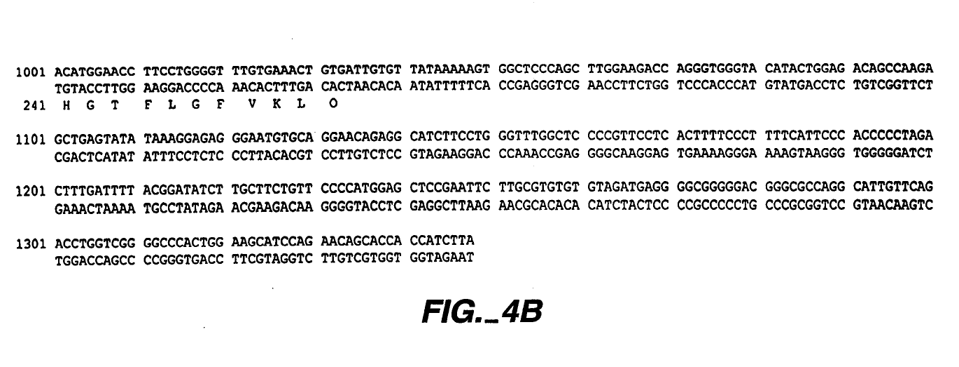

The terms “APRIL” or “APRIL polypeptide” when used herein encompass “native sequence APRIL polypeptides” and “APRIL variants”. “APRIL” is a designation given to those polypeptides which are encoded by the nucleic acid molecules comprising the polynucleotide sequences shown in FIG. 4A-4B and variants thereof, nucleic acid molecules comprising the sequence shown in the FIG. 4A-4B, and variants thereof as well as fragments of the above which have the biological activity of the native sequence APRIL. Variants of APRIL will preferably have at least 80%, more preferably, at least 90%, and even more preferably, at least 95% amino acid sequence identity with the native sequence APRIL polypeptide shown in FIG. 4A-4B. A “native sequence” APRIL polypeptide comprises a polypeptide having the same amino acid sequence as the corresponding APRIL polypeptide derived from nature. Such native sequence APRIL polypeptides can be isolated from nature or can be produced by recombinant and/or synthetic means. The term “native sequence APRIL polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide. The term “APRIL” includes those polypeptides described in Hahne et al., J. Exp. Med., 188:1185-1190 (1998); GenBank Accession No. AF046888; WO 99/00518 published Jan. 7, 1999; WO 99/35170 published Jul. 15, 1999; WO 99/12965 published Mar. 18, 1999; WO 99/33980 published Jul. 8, 1999; WO 97/33902 published Sep. 18, 1997; WO 99/11791 published Mar. 11, 1999; EP 911,633 published Mar. 28, 1999; and WO99/50416 published Oct. 7, 1999.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are identified by those that: (1) employ low ionic strength and high temperature for washing, 0.015 M sodium chloride/0.0015 M sodium citrate/0.1% sodium dodecyl sulfate at 50° C.; (2) employ during hybridization a denaturing agent, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” are identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “amino acid” and “amino acids” refer to all naturally occurring L-alpha-amino acids. This definition is meant to include norleucine, ornithine, and homocysteine. The amino acids are identified by either the single-letter or three-letter designations:

| | | | | | |
|---|---|---|---|---|---|
| Asp | D | aspartic acid | Ile | I | isoleucine |
| Thr | T | threonine | Leu | L | leucine |
| Ser | S | serine | Tyr | Y | tyrosine |
| Glu | E | glutamic acid | Phe | F | phenylalanine |
| Pro | P | proline | His | H | histidine |
| Gly | G | glycine | Lys | K | lysine |
| Ala | A | alanine | Arg | R | arginine |
| Cys | C | cysteine | Trp | W | tryptophan |
| Val | V | valine | Gln | Q | glutamine |
| Met | M | methionine | Asn | N | asparagine |

In the Sequence Listing and Figures, certain other single-letter or three-letter designations may be employed to refer to and identify two or more amino acids or nucleotides at a given position in the sequence.

“Percent (%) amino acid sequence identity” with respect to the ligand or receptor polypeptide sequences identified herein is defined as the percentage of amino acid residues in a candidate sequence that are identical with the amino acid residues in such a ligand or receptor sequence identified herein, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in the table below. The ALIGN-2 sequence comparison computer program was authored by Genentech, Inc. and the source code shown in the table below has been filed with user documentation in the U.S. Copyright Office, Washington D.C., 20559, where it is registered under U.S. Copyright Registration, No. TXU510087. The ALIGN-2 program is publicly available through Genentech, Inc., South San Francisco, Calif. or may be compiled from the source code provided in the table below. The ALIGN-2 program should be compiled for use on a UNIX operating system, preferably digital UNIX V4.0 D. All sequence comparison parameters are set by the ALIGN-2 program and do not vary.

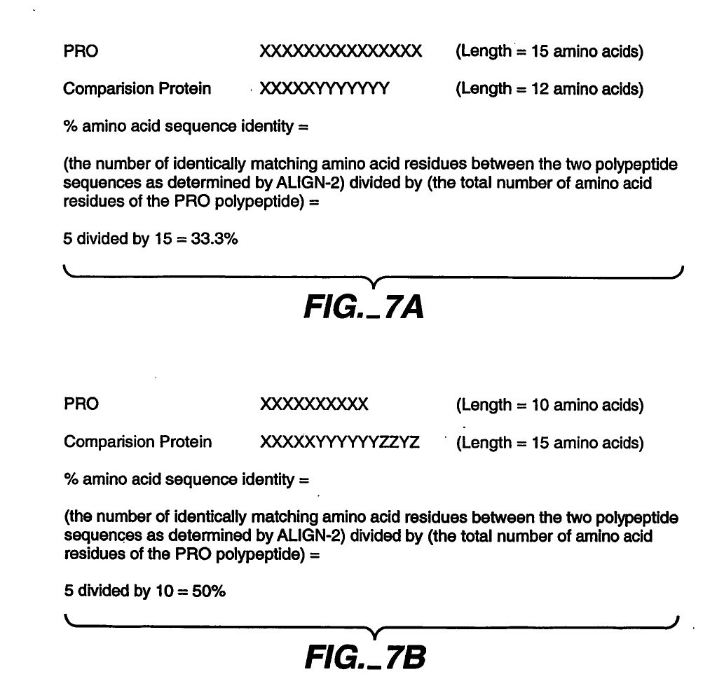

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, FIGS. 7A-7B demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from the NCBI internet web site. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to,.with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “epitope tagged” when used herein refers to a chimeric polypeptide comprising a polypeptide fused to a “tag polypeptide”. The tag polypeptide has enough residues to provide an epitope against which an antibody can be made. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The term “antagonist” is used in the broadest sense, and includes any molecule that partially or fully blocks, inhibits, or neutralizes one or more biological activities of TALL-1 polypeptide, APRIL polypeptide, or both TALL-1 and APRIL, in vitro, in situ, or In vivo. Examples of such biological activities of TALL-1 and APRIL polypeptides include binding of TALL-1 or APRIL to TACI, BCMA, TACIs or BR3, activation of NF-KB and activation of proliferation and of Ig secretion by B cells, immune-related conditions such as rheumatoid arthritis, as well as those further reported in the literature. An antagonist may function in a direct or indirect manner. For instance, the antagonist may function to partially or fully block, S inhibit or neutralize one or more biological activities of TALL-1 polypeptide, APRIL polypeptide, or both TALL-1 and APRIL, in vitro, in situ, or in vivo as a result of its direct binding to TALL-1, APRIL, BCMA, TACIs or BR3. The antagonist may also function indirectly to partially or fully block, inhibit or neutralize one or more biological activities of TALL-1 polypeptide, APRIL polypeptide, or both TALL-1 and APRIL, in vitro, in situ, or in vivo as a result of, e.g., blocking or inhibiting another effector molecule. The antagonist molecule may comprise a “dual” antagonist activity wherein the molecule is capable of partially or fully blocking, inhibiting or neutralizing a biological activity of both TALL-1 and APRIL.

The term “agonist” is used in the broadest sense, and includes any molecule that partially or fully enhances, stimulates or activates one or more biological activities of TACIs polypeptide, BR3 polypeptide, or both TACIs and BR3, in vitro, in situ, or in vivo. Examples of such biological activities of TACIs and BR3 may include activation of NF-KB, induction of immunoglobulin production and secretion, and cell proliferation. An agonist may function in a direct or indirect manner. For instance, the agonist may function to partially or fully enhance, stimulate or activate one or more biological activities of TACIs polypeptide, BR3 polypeptide, or both TACIs and BR3, in vitro, in situ, or in vivo as a result of its direct binding to TACIs or BR3, which causes receptor activation or signal transduction. The agonist may also function indirectly to partially or fully enhance, stimulate or activate one or more biological activities of TACIs polypeptide, BR3 polypeptide, or both TACIs and BR3, in vitro, in situ, or in vivo as a result of, e.g., stimulating another effector molecule which then causes TACIs or BR3 receptor activation or signal transduction. It is contemplated that an agonist may act as an enhancer molecule which functions indirectly to enhance or increase TACIs or BR3 activation or activity. For instance, the agonist may enhance activity of endogenous TALL-1 or APRIL in a mammal. This could be accomplished, for example, by pre-complexing TACIs or BR3 or by stabilizing complexes of the respective ligand with the TACIs or BR3 receptor (such as stabilizing native com0plex formed between TALL-1 and TACIs or APRIL and TACIs).

The term “TALL-1 antagonist” or “APRIL antagonist” refers to any molecule that partially or fully blocks, inhibits, or neutralizes a biological activity of TALL-1 or APRIL, respectively, or both TALL-1 and APRIL, and include, but are not limited to, soluble forms of TACIs receptor or BR3 receptor such as an extracellular domain sequence of TACIs or BR3, TACIs receptor immunoadhesins, BR3 receptor immunoadhesins, TACIs receptor fusion proteins, BR3 receptor fusion proteins, covalently modified forms of TACIs receptor, covalently modified forms of BR3 receptor, TACIs variants, BR3 variants, TACIs receptor antibodies, BR3 receptor antibodies, TALL-1 antibodies, and APRIL antibodies. To determine whether a TALL-1 antagonist molecule partially or fully blocks, inhibits or neutralizes a biological activity of TALL-1 or APRIL, assays may be conducted to assess the effect(s) of the antagonist molecule on, for example, binding of TALL-1 or APRIL to TACIs or to BR3, or NF-KB activation by the respective ligand. Such assays may be conducted in known in vitro or in vivo assay formats, for instance, in cells expressing BR3 and/or TACIs. Preferably, the TALL-1 antagonist employed in the methods described herein will be capable of blocking or neutralizing at least one type of TALL-1 activity, which may optionally be determined in assays such as described herein. To determine whether an APRIL antagonist molecule partially or fully blocks, inhibits or neutralizes a biological activity of TALL-1 or APRIL, assays may be conducted to assess the effect(s) of the antagonist molecule on, for example, binding of TALL-1 or APRIL to TACIs or to BR3, or NF-KB activation by the ligand. Such assays may be conducted in known in vitro or in vivo formats, for instance, using cells transfected with TACIs or BR3 (or both TACIs and BR3). Preferably, the APRIL antagonist employed in the methods described herein will be capable of blocking or neutralizing at least one type of APRIL activity, which may optionally be determined in a binding assay or an IgM-production assay. Optionally, a TALL-1 antagonist or APRIL antagonist will be capable of reducing or inhibiting binding of either TALL-1 or APRIL (or both TALL-1 and APRIL) to TACIs or to BR3 by at least 50%, preferably, by at least 90%, more preferably by at least 99%, and most preferably, by 100%, as compared to a negative control molecule, in a binding assay. In one embodiment, the TALL-1 antagonist or APRIL antagonist will comprise antibodies which will competitively inhibit the binding of another ligand or antibody to TACIs or BR3. Methods for determining antibody specificity and affinity by competitive inhibition are known in the art [see, e.g., Harlow et al., Antibodies:A Laboratory Manual, Cold Spring Harbor Laboratory Press, Cold Spring Harbor, N.Y. (1998); Colligan et al., Current Protocols in Immunology, Green Publishing Assoc., NY (1992; 1993); Muller, Meth. Enzym., 92:589-601 (1983).

The term “TACIs agonist” or “BR3 agonist” refers to any molecule that partially or fully enhances, stimulates or activates a biological activity of TACIs or BR3, respectively, or both TACIs and BR3, and include, but are not limited to, anti-TACIs receptor antibodies and anti-BR3 receptor antibodies. To determine whether a TACIs agonist molecule partially or fully enhances, stimulates, or activates a biological activity of TACIs or BR3, assays may be conducted to assess the effect(s) of the agonist molecule on, for example, PBLs or TACIs or BR3-transfected cells. Such assays may be conducted in known in vitro or in vivo assay formats. Preferably, the TACIs agonist employed in the methods described herein will be capable of enhancing or activating at least one type of TACIs activity, which may optionally be determined in assays such as described herein. To determine whether a BR3 agonist molecule partially or fully enhances, stimulates, or activates a biological activity of TACIs or BR3, assays may be conducted to assess the effect(s) of the agonist molecule on, for example, an activity of TALL-1 or BR3. Such assays may be conducted in in vitro or in vivo formats, for instance, using PBLs or BR3-transfected cells. Preferably, the TACIs agonist or BR3 agonist will be capable of stimulating or activating TACIs or BR3, respectively, to the extent of that accomplished by the native ligand(s) for the TACIs or BR3 receptors.

The term “antibody” is used in the broadest sense and specifically covers, for example, single monoclonal antibodies against BR3, TACIs, TALL-1, APRIL, TACI, or BCMA, antibody compositions with polyepitopic specificity, single chain antibodies, and fragments of antibodies. “Antibody” as used herein includes intact immunoglobulin or antibody molecules, polyclonal antibodies, multispecific antibodies (i.e., bispecific antibodies formed from at least two intact antibodies) and immunoglobulin fragments (such as Fab, F(ab′)₂, or Fv), so long as they exhibit any of the desired agonistic or antagonistic properties described herein.

Antibodies are typically proteins or polypeptides which exhibit binding specificity to a specific antigen. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains [Chothia et al., J. Mol. Biol., 186:651-663 (1985); Novotny and Haber, Proc. Natl. Acad. Sci. USA, 82:4592-4596 (1985)]. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

“Antibody fragments” comprise a portion of an intact antibody, generally the antigen binding or variable region of the intact antibody. Examples of antibody fragments include Fab, Fab′, F(ab′)2, and Fv fragments, diabodies, single chain antibody molecules; and multispecific antibodies formed from antibody fragments.

The term “variable” is used herein to describe certain portions of the variable domains which differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies [see Kabat, E. A. et al., Sequences of Proteins of Immunological Interest, National Institutes of Health, Bethesda, Md. (1987)]. The constant domains are not involved directly in binding an antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include chimeric, hybrid and recombinant antibodies produced by splicing a variable (including hypervariable) domain of the antibody of interest with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity or properties. See, e.g. U.S. Pat. No. 4,816,567 and Mage et al., in Monoclonal Antibody Production Techniques and Applications, pp.79-97 (Marcel Dekker, Inc.: New York, 1987).

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

“Humanized” forms of non-human (e.g. murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

A “human antibody” is one which possesses an amino acid sequence which corresponds to that of an antibody produced by a human and/or has been made using any of the techniques for making human antibodies known in the art or as disclosed herein. This definition of a human antibody includes antibodies comprising at least one human heavy chain polypeptide or at least one human light chain polypeptide, for example an antibody comprising murine light chain and human heavy chain polypeptides. Human antibodies can be produced using various techniques known in the art. In one embodiment, the human antibody is selected from a phage library, where that phage library expresses human antibodies (Vaughan et al. Nature Biotechnology, 14:309-314 (1996): Sheets et al. PNAS, (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10: 779-783 (1992); Lonberg et al., Nature, 368: 856-859 (1994); Morrison, Nature, 368:812-13 (1994); Fishwild et al., Nature Biotechnology, 14: 845-51 (1996); Neuberger, Nature Biotechnology, 14: 826 (1996); Lonberg and Huszar, Intern. Rev. Immunol., 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region (using herein the numbering system according to Kabat et al., supra). The Fc region of an immunoglobulin generally comprises two constant domains, a CH2 domain and a CH3 domain, and optionally comprises a CH4 domain.

By “Fc region chain” herein is meant one of the two polypeptide chains of an Fc region.

The “CH2 domain” of a human IgG Fc region (also referred to as “Cγ2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol.22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol.22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of a Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA), 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” and “FcR” are used to describe a receptor that binds to the Fc region of an antibody. The preferred FcR is a native sequence human FcR. Moreover, a, preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol., 117:587 (1976); and Kim et al., J. Immunol., 24:249 (1994)).

“Complement dependent cytotoxicity” and “CDC” refer to the lysing of a target in the presence of complement. The complement activation pathway is initiated by the binding of the first component of the complement system (C1q) to a molecule (e.g. an antibody) completed with a cognate antigen. To assess complement activation, a CDC assay, e.g. as described in Gazzano-Santoro et al., J. Immunol. Methods, 202:163 (1996), may be performed.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology, 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene, 169:147-155 (1995); Yelton et al. J. Immunol., 155:1994-2004 (1995); Jackson et al., J. Immunol., 154(7):3310-9 (1995); and Hawkins et al, J. Mol. Biol., 226:889-896 (1992).

The term “immunospecific” as used in “immunospecific binding of antibodies” for example, refers to the antigen specific binding interaction that occurs between the antigen-combining site of an antibody and the specific antigen recognized by that antibody.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes protein in situ within recombinant cells, since at least one component of the protein natural environment will not be present. Ordinarily, however, isolated protein will be prepared by at least one purification step.

“Treatment” or “therapy” refer to both therapeutic treatment and prophylactic or preventative measures.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

“TALL-1-related pathological condition” and “APRIL-related pathological condition” refer to pathologies or conditions associated with abnormal levels of expression or activity of TALL-1 or APRIL, respectively, in excess of, or less than, levels of expression or activity in normal healthy mammals, where such excess or diminished levels occur in a systemic, localized, or particular tissue or cell type or location in the body. TALL-1-related pathological conditions and APRIL-related pathological conditions include acute and chronic immune related diseases and cancer.

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Optionally, the cancer will express, or have associated with the cancer cell, TALL-1, APRIL, TACI, TACIs, BR3 or BCMA. By way of example, colon, lung and melanoma cancers have been reported in the literature to express APRIL. The preferred cancers for treatment herein include lymphoma, leukemia and myeloma, and subtypes thereof, such as Burkitt's lymphoma, multiple myeloma, acute lymphoblastic or lymphocytic leukemia, non-Hodgkin's and Hodgkin's lymphoma, and acute myeloid leukemia.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

“Autoimmune disease” is used herein in a broad, general sense to refer to disorders or conditions in mammals in which destruction of normal or healthy tissue arises from humoral or cellular immune responses of the individual mammal to his or her own tissue constituents. Examples include, but are not limited to, lupus erythematous, thyroiditis, rheumatoid arthritis, psoriasis, multiple sclerosis, autoimmune diabetes, and inflammatory bowel disease (IBD).

The term “prodrug” as used in this application refers to a precursor or derivative form of a pharmaceutically active substance that is less cytotoxic to cancer cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described below.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³₁, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of conditions like cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin Y₁^{I} and calicheamicin θ^{I}₁, see, e.g., Agnew Chem Intl. Ed. Engl. 33:183-186 (1994); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERP®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (W B Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors; platelet-growth factor; transforming growth factors (TGFs) such as TGF-α and TGF-β; insulin-like growth factor-I and -II; erythropoietin (EPO); osteoinductive factors; interferons such as interferon-α, -β, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-11, IL-12; and other polypeptide factors including LIF and kit ligand (KL). As used herein, the term cytokine includes proteins from natural sources or from recombinant cell culture and biologically active equivalents of the native sequence cytokines.

II. Methods and Materials

The present invention provides newly identified and isolated nucleotide sequences encoding polypeptides referred to in the present application as TACIs and BR3. In particular, Applicants have identified and isolated cDNA encoding TACIs polypeptides and encoding BR3 polypeptides, as disclosed in further detail in the Examples below.

### A. Variants of the TACIs and BR3 Polypeptides

In addition to the full-length native sequence TACIs polypeptides and BR3 polypeptides described herein, it is contemplated that respective polypeptide variants can be prepared. Polypeptide variants can be prepared by introducing appropriate nucleotide changes into the TACIs- or BR3-polypeptide-encoding DNA, or by synthesis of the desired TACIs or BR3 polypeptide. Those skilled in the art will appreciate that amino acid changes may alter post-translational processes of the polypeptide, such as changing the number or position of glycosylation sites or altering the membrane anchoring characteristics.

Variations in the native full-length sequence polypeptide or in various domains of the polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the TACIs or BR3 polypeptide that results in a change in the amino acid sequence of the TACIs or BR3 polypeptide as compared with the native sequence polypeptide. Optionally, the variation is by substitution of at least one amino acid with any other amino acid in one or more of the domains of the TACIs or BR3 polypeptide.

Guidance in determining which amino acid residue may be inserted, substituted or deleted without adversely affecting the desired activity may be found by comparing the sequence of the polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the TACIs polypeptide or BR3 polypeptide-encoding variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isosteric amino acid can be used.

### B. Modifications of the TACIs or BR3 Polypeptides

Covalent modifications of TACIs polypeptides or of BR3 polypeptides are included within the scope of this invention. N. terminal methionine residues may be present or absent on the polypeptides disclosed herein. One type of covalent modification includes reacting targeted amino acid residues of a TACIs polypeptide with an organic derivatizing agent that is capable of reacting with selected side chains or the N- or C- terminal residues of a TACIs polypeptide. A BR3 polypeptide can be similarly modified at targeted amino acid residues having selected side chains or at its N- or C-terminal residues.

Derivatization with bifunctional agents is useful, for instance, for crosslinking TACIs polypeptide to a water-insoluble support matrix or surface for use in the method for purifying anti-TACIs polypeptide antibodies, and vice-versa. Such bifunctional agents are also useful for crosslinking BR3 polypeptide to a water-insoluble support matrix or surface for use in the method for purifying anti-BR3 polypeptide antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis-(succinimidylpropionate), bifunctional maleimides such as bis-N-maleimido-1,8-octane and agents such as methyl-3-[(p-azidophenyl)-dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the TACIs polypeptide or BR3 polypeptide included within the scope of this invention comprises altering the native glycosylation pattern of either polypeptide. “Altering the native glycosylation pattern” is intended for purposes herein to mean deleting one or tore carbohydrate moieties found in native sequence TACIs polypeptide, deleting one or more carbohydrate moieties found in native sequence BR3 polypeptide, adding one or more glycosylation sites that are not present in the native sequence TACIs polypeptide, and/or adding one or more glycosylation sites that are not present in the native sequence BR3 polypeptide.

Addition of glycosylation sites to TACIs polypeptides or BR3 polypeptides may be accomplished by altering the amino acid sequence thereof. The alteration may be made, for example, by the addition of, or substitution by, one or more serine or threonine residues to the native sequence TACIs polypeptide, or one or more serine or threonine residues to the native sequence BR3 polypeptide (for O-linked glycosylation sites). The TACIs polypeptide amino acid sequence may optionally be altered through changes at the DNA level, particularly by mutating the DNA encoding the TACIs polypeptide at preselected bases such that codons are generated that will translate into the desired amino acids. Similarly, the BR3 polypeptide amino acid sequence may optionally be altered through changes at the DNA level, particularly by mutating the DNA encoding the BR3 polypeptide at preselected bases such that codons are generated that will translate into the desired amino acids.

Another means of increasing the number of carbohydrate moieties on the TACIs polypeptide or BR3 polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the TACIs polypeptide or BR3 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of TACIs polypeptide or BR3 polypeptide comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

### C. Preparation of TACIs and BR3 Polypeptides

The description below relates primarily to production of a polypeptide, such as TACIs polypeptide, by culturing cells transformed or transfected with a vector containing TACIs polypeptide encoding nucleic acid. It is, of course, contemplated that alternative methods, which are well known in the art, may be employed to prepare TACIs polypeptides. For instance, the TACIs polypeptide sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of TACIs polypeptides may be chemically synthesized separately and combined using chemical or enzymatic methods to produce a full-length TACIs polypeptide.

The description below also relates to production of BR3 polypeptide by culturing cells transformed or transfected with a vector containing BR3 polypeptide encoding nucleic acid. It is, of course, contemplated that alternative methods, which are well known in the art, may be employed to prepare BR3 polypeptides. For instance, the BR3 polypeptide sequence, or portions thereof, may be produced by direct peptide synthesis using solid-phase techniques, as described above. Various portions of BR3 polypeptides may be chemically synthesized separately and combined using chemical or enzymatic methods to produce a full-length BR3 polypeptide.

### 1. Isolation of DNA Encoding TACIs or BR3 Polypeptides

DNA encoding a TACIs polypeptide may be obtained from a cDNA library prepared from tissue believed to possess the TACIs polypeptide mRNA and to express it at a detectable level. Accordingly, human TACIs polypeptide-encoding DNA can be conveniently obtained from a cDNA library prepared from human tissue. The TACIs polypeptide-encoding gene may also be obtained from a genomic library or by oligonucleotide synthesis.

Similarly, DNA encoding a BR3 polypeptide may be obtained from a cDNA library prepared from tissue believed to possess the BR3 polypeptide mRNA and to express it at a detectable level. Accordingly, human BR3 polypeptide-encoding DNA can be conveniently obtained from a cDNA library prepared from human tissue. The BR3 polypeptide-encoding gene may also be obtained from a genomic library or by oligonucleotide synthesis.

Libraries can be screened with probes (such as antibodies to a TACIs polypeptide, antibodies to a BR3 polypeptide, or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding TACIs polypeptide or the gene encoding BR3 polypeptide is to use PCR methodology [Sambrook et al., supra; Dieffenbach et al., PCR Primer:A Laboratory Manual (Cold Spring Harbor Laboratory Press, 1995)].

In techniques for screening a cDNA library, the oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra, and are defined above. Optionally, the hybridizations conditions are high stringency as defined on page 22, lines 6-20.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs such as those referred to above, and optionally using the ALIGN-2 program provided herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2. Selection and Transformation of Host Cells

Host cells are transfected or transformed with expression or cloning vectors described herein for TACIs polypeptide production. Alternatively, host cells are transfected or transformed with expression or cloning vectors described herein for BR3 polypeptide production. The host cells are cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for vectors encoding TACIs polypeptide or vectors encoding BR3 polypeptide. Saccharomyces cerevisiae is a commonly used lower eukaryotic host microorganism.

Suitable host cells for the expression of glycosylated TACIs polypeptide or of glycosylated BR3 polypeptide are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### 3. Selection and Use of a Replicable Vector

The nucleic acid (e.g., cDNA or genomic DNA) encoding the desired TACIs polypeptide or encoding the desired BR3 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The desired TACIs polypeptide or the desired BR3 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, it may be a part of the TACIs polypeptide-encoding DNA that is inserted into the vector, or it may be a part of the BR3 polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the TACIs polypeptide-encoding nucleic acid or the BR3 polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the TACIs polypeptide-encoding nucleic acid sequence or to the BR3 polypeptide-encoding nucleic acid sequence. The promoter directs mRNA synthesis. Promoters recognized by a variety of potential host cells are well known. Promoters suitable for use with prokaryotic hosts include the beta-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding the polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

TACIs polypeptide or BR3 polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription by higher eukaryotes of a DNA encoding a TACIs polypeptide or of a DNA encoding a BR3 polypeptide may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the TACIs polypeptide coding sequence, but is preferably located at a site 5′ from the promoter. Similarly, the enhancer may be spliced into the vector at a position 5′ or 3′ to the BR3 polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding TACIs polypeptide or of the mRNA encoding BR3 polypeptide.

Still other methods, vectors, and host cells suitable for adaptation to the synthesis of TACIs polypeptides and/or BR3 polypeptides in recombinant vertebrate cell culture are described in Gething et al., Nature, 293:620-625 (1981); Mantei et al., Nature, 281:40-46 (1979); EP 117,060; and EP 117,058.

### 4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence TACIs polypeptide, against a native sequence BR3 polypeptide, against a synthetic peptide based on the DNA sequences provided herein, against an exogenous sequence fused to TACIs polypeptide-encoding DNA and encoding a specific antibody epitope, or against an exogenous sequence fused to BR3 polypeptide-encoding DNA and encoding a specific antibody epitope.

### 5. Polypeptide Purification

Forms of TACIs polypeptide or BR3 polypeptide may be recovered from culture medium or from host cell lysates. If membrane-bound, they can be released from the membrane using a suitable detergent solution (e.g. Triton-X 100) or by enzymatic cleavage. Cells employed in expression of TACIs polypeptides or BR3 polypeptides can be disrupted by various physical or chemical means, such as freeze-thaw cycling, sonication, mechanical disruption, or cell lysing agents.

It may be desired to purify TACIs polypeptide or BR3 polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the TACIs polypeptide or BR3 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular TACIs polypeptide or BR3 polypeptide produced.

### 6. Uses for TACIs Polypeptide or BR3 Polypeptide

Nucleotide sequences (or their complement) encoding TACIs polypeptides, and nucleotide sequences or their complements encoding BR3 polypeptides, have various applications in the art of molecular biology, including uses as hybridization probes, in chromosome and gene mapping and in the generation of anti-sense RNA and DNA. TACIs polypeptide-encoding nucleic acid will also be useful for the preparation of TACIs polypeptides by the recombinant techniques described herein. Similarly, BR3 polypeptide-encoding nucleic acid will also be useful for the preparation of BR3 polypeptides by the recombinant techniques described herein.

Nucleic acids which encode TACIs polypeptide, BR3 polypeptide, or any of their modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding TACIs polypeptide can be used to clone genomic DNA encoding TACIs polypeptide in accordance with established techniques and the genomic sequences used to generate transgenic animals that contain cells which express DNA encoding TACIs polypeptide. In another embodiment, cDNA encoding BR3 polypeptide can be used to clone genomic DNA encoding BR3 polypeptide in accordance with established techniques and the genomic sequences used to generate transgenic animals that contain cells which express DNA encoding BR3 polypeptide.

Methods for generating transgenic animals, particularly animals such as mice or rats, have become conventional in the art and are described, for example, in U.S. Pat. Nos. 4,736,866 and 4,870,009. Typically, particular cells would be targeted for TACIs polypeptide and/or BR3 polypeptide transgene incorporation with tissue-specific enhancers. Transgenic animals that include a copy of a transgene encoding TACIs polypeptide introduced into the germ line of the animal at,an embryonic stage can be used to examine the effect of increased expression of DNA encoding TACIs polypeptide. Alternatively, transgenic animals that include a copy of a transgene encoding BR3 polypeptide introduced into the germ line of the animal at an embryonic stage can be used to examine the effect of increased expression of DNA encoding BR3 polypeptide. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of TACIs polypeptide can be used to construct a TACIs polypeptide “knock out” animal which has a defective or altered gene encoding TACIs polypeptide as a result of homologous recombination between the endogenous gene encoding TACIs polypeptide and altered genomic DNA encoding TACIs polypeptide introduced into an embryonic cell of the animal. For example, cDNA encoding TACIs polypeptide can be used to clone genomic DNA encoding TACIs polypeptide in accordance with established techniques. A portion of the genomic DNA encoding TACIs polypeptide can be deleted or replaced with another gene, such as a gene encoding a selectable marker which can be used to monitor integration.

Similarly, non-human homologues of BR3 polypeptide can be used to construct a BR3 polypeptide “knock out” animal which has a defective or altered gene encoding BR3 polypeptide as a result of homologous recombination between the endogenous gene encoding BR3 polypeptide and altered genomic DNA encoding BR3 polypeptide introduced into an embryonic cell of the animal. For example, cDNA encoding BR3 polypeptide can be used to clone genomic DNA encoding BR3 polypeptide in accordance with established techniques. A portion of the genomic DNA encoding BR3 polypeptide can be deleted or replaced with another gene, such as a gene encoding a selectable marker which can be used to monitor integration.

Typically, in constructing a “knock out animal”, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected (see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knock out animals can be characterized for instance, for their ability to defend against certain pathological conditions and for their development of pathological conditions due to absence of the TACIs polypeptide or the BR3 polypeptide.

The TACIs polypeptide or the BR3 polypeptide herein may be employed in accordance with the present invention by expression of such polypeptides in vivo, which is often referred to as gene therapy.

There are two major approaches to getting the nucleic acid (optionally contained in a vector) into the patient's cells: in vivo and ex vivo. For in vivo delivery the nucleic acid is injected directly into the patient, usually at the sites where the polypeptide is required. For example, TACIs polypeptide-encoding nucleic acid will be injected at the site of synthesis of the TACIs polypeptide, if known, or the site where biological activity of TACIs polypeptide is needed. For example, BR3 polypeptide-encoding nucleic acid will be injected at the site of synthesis of the BR3 polypeptide, if known, or the site where biological activity of BR3 polypeptide is needed. For ex vivo treatment, the patient's cells are removed, the nucleic acid is introduced into these isolated cells, and the modified cells are administered to the patient either directly or, for example, encapsulated within porous membranes that are implanted into the patient (see, e.g., U.S. Pat. Nos. 4,892,538 and 5,283,187).

There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, transduction, cell fusion, DEAE-dextran, the calcium phosphate precipitation method, etc. Transduction involves the association of a replication-defective, recombinant viral (preferably retroviral) particle with a cellular receptor, followed by introduction of the nucleic acids contained by the particle into the cell. A commonly used vector for ex vivo delivery of the gene is a retrovirus.

The currently preferred in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinson et al., Cancer Investigation, 14(1): 54-65 (1996)). The most preferred vectors for use in gene therapy are viruses, most preferably adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding TACIs polypeptide or of a gene encoding BR3 polypeptide, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the TACIs polypeptide or BR3 polypeptide from a host cell in which it is placed. Preferably the signal sequence for this purpose is a mammalian signal sequence, most preferably the native signal sequence for TACIs polypeptide or for BR3 polypeptide. optionally, the vector construct may also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be-used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414 (1990). For a review of the currently known gene marking and gene therapy protocols, see Anderson et al., Science, 256: 808-813 (1992). See also WO 93/25673 and the references cited therein. Suitable gene therapy and methods for making retroviral particles and structural proteins can be found in, e.g., U.S. Pat. No. 5,681,746.

The invention further provides methods for modulating TALL-1, APRIL, TACI, BCMA, TACIs, and/or BR3 activity in mammalian cells which comprise exposing the cells to a desired amount of antagonist or agonist that affects TALL-1 or APRIL interaction with TACI, BCMA, TACIs or BR3. Preferably, the amount of antagonist or agonist employed will be an amount effective to affect the binding and/or activity of the respective ligand or respective receptor to achieve a therapeutic effect. This can be accomplished in vivo or ex vivo in accordance, for instance, with the methods described below and in the Examples. Exemplary conditions or disorders to be treated with such TALL-1 antagonists or APRIL antagonists include conditions in mammals clinically referred to as autoimmune diseases, including but not limited to rheumatoid arthritis, multiple sclerosis, psoriasis, and lupus or other pathological conditions in which B cell response(s) in mammals is abnormally upregulated such as cancer. Exemplary conditions or disorders to be treated with TACIs agonists or BR3 agonists include immunodeficiency and cancer.

Diagnostic methods are also provided herein. For instance, the antagonists or agonists may be employed to detect the respective ligands (TALL-1 or APRIL) or receptors (TACIs or BR3) in mammals known to be or suspected of having a TALL-1—related pathological condition or APRIL-related pathological condition. The antagonist or agonist molecule may be used, e.g., in immunoassays to detect or quantitate TALL-1 or APRIL in a sample. A sample, such as cells obtained from a mammal, can be incubated in the presence of a labeled antagonist or agonist molecule, and detection of the labeled antagonist or agonist bound in the sample can be performed. Such assays, including various clinical assay procedures, are known in the art, for instance as described in Voller et al., Immunoassays, University Park, 1981.

The antagonists and agonists which can be employed in the methods include, but are not limited to, soluble forms of TACIs and BR3 receptors, TACIs receptor immunoadhesins and BR3 receptor immunoadhesins, fusion proteins comprising TACIs or BR3, covalently modified forms of TACIs or BR3, TACIs receptor variants and BR3 receptor variants, TACIs or BR3 receptor antibodies, and TALL-1 or APRIL antibodies. Various techniques that can be employed for making the antagonists and agonists are described herein. For instance, methods and techniques for preparing TACIs and BR3 polypeptides are described above. Below, further modifications of the polypeptides, and antibodies to TACIs and BR3 are described.

Soluble forms of TACIs receptors or BR3 receptors may be employed as antagonists in the methods of the invention. Such soluble forms of TACIs or BR3 may comprise or consist of extracellular domains of the respective receptor (and lacking transmembrane and intracellular domains of the respective receptor). The extracellular domain sequences themselves of TACIs or BR3 may be used as antagonists, or may be further modified as described below (such as by fusing to an immunoglobulin, epitope tag or leucine zipper). Those skilled in the art will be able to select, without undue experimentation, a desired extracellular domain sequence of either TACIs or BR3 to employ as an antagonist.

Immunoadhesin molecules are further contemplated for use in the methods herein. TACIs receptor immunoadhesins may comprise various forms of TACIs, such as the full length polypeptide as well as soluble forms of the receptor which comprise an extracellular domain (ECD) sequence or a fragment of the ECD sequence. In one embodiment, the molecule may comprise a fusion of the TACIs receptor with an immunoglobulin or a particular region of an immunoglobulin. For a bivalent form of the immunoadhesin, such a fusion could be to the Fc region of an IgG molecule. The Ig fusions preferably include the substitution of a soluble (transmembrane domain deleted or inactivated) form of the receptor polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

The simplest and most straightforward immunoadhesin design combines the binding domain(s) of the adhesin (e.g. the extracellular domain (ECD) of a receptor) with the Fc region of an immunoglobulin heavy chain ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:
(a) AC_{L}-AC_{L};
(b) AC_{H}-(AC_{H}, AC_{L}-AC_{H}, AC_{L}—V_{H}C_{H}, or V_{L}C_{L}-AC_{H});
(c) AC_{L}-AC_{H}-(AC_{L}-AC_{H}, AC_{L}—V_{H}C_{H}, V_{L}C_{L}-AC_{H}, or V_{L}C_{L}—VₕC_{H})
(d) AC_{L}—V_{H}C_{H}-(AC_{H}, or AC_{L}—V_{H}C_{H}, or V_{L}C_{L}-AC_{H});
(e) V_{L}C_{L}-AC_{H}-(AC_{L}—V_{H}C_{H}, or V_{L}C_{L}-AC_{H}); and
(f) (A-Y)ₙ-(V_{L}C_{L}-V_{H}C_{H})₂,
   wherein each A represents identical or different adhesin amino acid sequences;
V_{L} is an immunoglobulin light chain variable domain;
V_{H} is an immunoglobulin heavy chain variable domain;
C_{L} is an immunoglobulin light chain constant domain;
C_{H} is an immunoglobulin heavy chain constant domain;
n is an integer greater than 1;
Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}² and C_{H}3 domains. Similar constructs have been-reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Examples of such soluble ECD sequences include polypeptides comprising amino acids 1 to 119 of the TACIs sequence shown in FIG. 5B. The TACIs receptor immunoadhesin can be made according to any of the methods described in the art.

BR3 receptor immunoadhesins can be similarly constructed. Examples of soluble ECD sequences for use in constructing BR3 immunoadhesins may include polypeptides comprising amino acids 1 to 77 or 2 to 62 of the BR3 sequence shown in FIG. 6B.

In another embodiment, the TACIs or BR3 receptor may be covalently modified by linking the receptor polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. Such pegylated forms of the TACIs or BR3 receptor may be prepared using techniques known in the art.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the TACIs or BR3 receptor molecule.

The TACIs or BR3 polypeptides of the present invention may also be modified in a way to form chimeric molecules by fusing the receptor polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the TACIs or BR3 receptor polypeptide with a tag polypeptide which provides an epitope to which an anti-tag antibody can selectively bind. The epitope tag is generally placed at the amino- or carboxyl-terminus of the receptor polypeptide. The presence of such epitope-tagged forms of the receptor can be detected using an antibody against the tag polypeptide. Also, provision of the epitope tag enables the receptor to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

It is contemplated that anti-TACIs receptor antibodies or anti-BR3 antibodies may also be employed in the presently disclosed methods. Examples of such molecules include neutralizing or blocking antibodies which can preferably inhibit binding of TALL-1 or APRIL to the TACIs or to the BR3 receptors. The anti-TACIs antibodies or anti-BR3 antibodies may be monoclonal antibodies.

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include a TACIs or BR3 polypeptide (or a TACIs ECD or BR3 ECD) or a fusion protein thereof, such as a TACIs ECD-IgG fusion protein. The immunizing agent may alternatively comprise a fragment or portion of TACIs or BR3 having one or more amino acids that participate in the binding of TALL-1 or APRIL to TACIs or BR3. In a preferred embodiment, the immunizing agent comprises an extracellular domain sequence of TACIs or BR3 fused to an IgG sequence.

Generally, either peripheral blood lymphocytes (“PBLs”) are used if cells of human origin are desired, or spleen cells or lymph node cells are used if non-human mamalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against TACIs or BR 3. Preferably, the binding specificity of monoclonal antibodies produced by the hybridoma cells is determined by immunoprecipitation or by an In vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. 81, 6851 (1984), or by covalently joining to the immunoglobulin coding sequence all or part of the coding sequence for a non-immunoglobulin polypeptide.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for TACIs or BR3 and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., FEBS Letters, 409:437-441 (1997). Coupling of such single chain fragments using various linkers is described in Kortt et al., Protein Engineering, 10:423-433 (1997). A variety of techniques for the recombinant production and manipulation of antibodies are well known in the art. Illustrative examples of such techniques that are typically utilized by skilled artisans are described in greater detail below.

(i) Humanized Antibodies Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody.

Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

(ii) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pp.51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Mendez et al. (Nature Genetics 15: 146-156 [1997]) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Alternatively, the phage display technology (McCafferty et al., Nature 348, 552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nucl. Acids Res. 21, 2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published 1 Apr. 1993). Unlike traditional humanization of rodent antibodies by CDR grafting, this technique provides completely human antibodies, which have no framework or CDR residues of rodent origin.

As discussed below, the antibodies of the invention may optionally comprise monomeric, antibodies, dimeric antibodies, as well as multivalent forms of antibodies. Those skilled in the art may. construct such dimers or multivalent forms by techniques known in the art. Methods for preparing monovalent antibodies are also well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

(iii) Bispecific Antibodies

Bispecific antibodies are monoclonal, preferably human or humanized, antibodies that have binding specificities for at least two different antigens. In the present case, one of the binding specificities is for the TACIs or BR3 receptor, the other one is for any other antigen, and preferably for another receptor or receptor subunit. For example, bispecific antibodies specifically binding a TACIs or BR3 receptor and another apoptosis/signalling receptor are within the scope of the present invention.

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published 13 May 1993), and in Traunecker et al., EMBO 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690, published on Mar. 3, 1994.

For further details of generating bispecific antibodies see, for example, Suresh et al., Methods in Enzymology 121, 210 (1986).

(iv) Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

(v) Antibody Fragments

In certain embodiments, the anti-TACIs or anti-BR3 antibody (including murine, human and humanized antibodies, and antibody variants) is an antibody fragment. Various techniques have been developed for the production of antibody fragments. Traditionally, these fragments were derived via proteolytic digestion of intact antibodies (see, e.g., Morimoto et al., J. Biochem, Biophys. Methods 24:107-117 (1992) and Brennan et al., Science 229:81 (1985)). However, these fragments can now be produced directly by recombinant host cells. For example, Fab′-SH fragments can be directly recovered from E. coli and chemically coupled to form F(ab′)₂ fragments (Carter et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. A variety of techniques for the production of antibody fragments will be apparent to the skilled practitioner. For instance, digestion can be performed using papain. Examples of papain digestion are described in WO 94/29348 published 12/22/94 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Antibodies are glycosylated at conserved positions in their constant regions (Jefferis and Lund, Chem. Immunol. 65:111-128 [1997]; Wright and Morrison, TibTECH 15:26-32 [1997)). The oligosaccharide side chains of the immunoglobulins affect the protein's function (Boyd et al., Mol. Immunol. 32:1311-1318 [1996]; Wittwe and Howard, Biochem. 29:4175-4180 [1990]), and the intramolecular interaction between portions of the glycoprotein which can affect the conformation and presented three-dimensional surface of the glycoprotein (Hefferis and Lund, supra; Wyss and Wagner, Current Opin. Biotech. 7:409-416 [1996)). Oligosaccharides may also serve to target a given glycoprotein to certain molecules based upon specific recognition structures.: For example, it has been reported that in agalactosylated IgG, the oligosaccharide moiety ‘flips’ out of the inter-CH2 space and terminal N-acetylglucosamine residues become available to bind mannose binding protein (Malhotra et al., Nature Med. 1:237-243 [1995]). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., Mol. Immunol. 32:1311-1318 [1996]), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180 [1999]).

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation (including glycosylation pattern) of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, significant variations in the glycosylation pattern of the antibodies can be expected (see, e.g. Hse et al., J. Biol. Chem. 272:9062-9070 [1997]). In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5.278,299). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

Triabodies are also within the scope of the invention. Such antibodies are described for instance in Iliades et al., supra and Kortt et al., supra.

The antibodies of the present invention may be modified by conjugating the antibody to a cytotoxic agent (like a toxin molecule) or a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278. This technology is also referred to as “Antibody Dependent Enzyme Mediated Prodrug Therapy” (ADEPT).

The enzyme component of the immunoconjugate useful for ADEPT includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; caspases such as caspase-3; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes can be covalently bound to the antibodies by techniques well known in the art such as the use of heterobifunctional crosslinking reagents. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

Further antibody modifications are contemplated. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### D. Assay Methods

Ligand/receptor binding studies may be carried out in any known assay method, such as competitive binding assays, direct and indirect sandwich assays, and immunoprecipitation assays. Cell-based assays and animal models can be used as diagnostic methods and to further understand the interaction between the ligands and receptors identified herein and the development and pathogenesis of the conditions and diseases referred to herein.

In one approach, mammalian cells may be transfected with the ligands or receptors described herein, and the ability of the agonists or antagonists to stimulate or inhibit binding or activity is analyzed. Suitable cells can be transfected with the desired gene, and monitored for activity. Such transfected cell lines can then be used to test the ability of antagonist(s) or agonist(s) to inhibit or stimulate, for example, to modulate B-cell proliferation or Ig secretion. Cells transfected with the coding sequence of the genes identified herein can further be used to identify drug candidates for the treatment of immune related diseases or cancer.

In addition, primary cultures derived from transgenic animals can be used in the cell-based assays. Techniques to derive continuous cell lines from transgenic animals are well known in the art. [see, e.g., Small et al., Mol. Cell. Biol., 5:642-648 (1985)].

One suitable cell based assay is the addition of epitope-tagged ligand (e.g., AP or Flag) to cells that have or express the respective receptor, and analysis of binding (in presence or absence or prospective antagonists) by FACS staining with anti-tag antibody. In another assay, the ability of an antagonist to inhibit the TALL-1 or APRIL induced proliferation of B cells is assayed. B cells or cell lines are cultured with TALL-1 or APRIL in the presence or absence or prospective antagonists and the proliferation of B cells can be measured by ³H-thymidine incorporation or cell number.

The results of the cell based in vitro assays can be further verified using in vivo animal models. A variety of well known animal models can be used to further understand the role of the agonists and antagonists identified herein in the development and pathogenesis of for instance, immune related disease or cancer, and to test the efficacy of the candidate therapeutic agents. The in vivo nature of such models makes them particularly predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule.

Animal models, for example, for graft-versus-host disease are known. Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing T cell reactivity against MHC antigens and minor transplant antigens. A suitable procedure is described in detail in Current Protocols in Immunology, unit 4.3.

An animal model for skin allograft rejection is a means of testing the ability of T cells to mediate in vivo tissue destruction which is indicative of and a measure of their role in anti-viral and tumor immunity. The most common and accepted models use murine tail-skin grafts. Repeated experiments have shown that skin allograft rejection is mediated by T cells, helper T cells and killer-effector T cells, and not antibodies. [Auchincloss, H. Jr. and Sachs, D. H., Fundamental Immunology, 2nd ed., W. E. Paul ed., Raven Press, NY, 1989, 889-992]. A suitable procedure is described in detail in Current Protocols in Immunology, unit 4.4. Other transplant rejection models which can be used to test the compositions of the invention are the allogeneic heart transplant models described by Tanabe, M. et al., Transplantation, (1994) 58:23 and Tinubu, S. A. et al., J. Immunol., (1994) 4330-4338.

Animal models for delayed type hypersensitivity provides an assay of cell mediated immune function as well. Delayed type hypersensitivity reactions are a T cell mediated in vivo immune response characterized by inflammation which does not reach a peak until after a period of time has elapsed after challenge with an antigen. These reactions also occur in tissue specific autoimmune diseases such as multiple sclerosis (MS) and experimental autoimmune encephalomyelitis (EAE, a model for MS). A suitable procedure is described in detail in Current Protocols in Immunology, unit 4.5.

An animal model for arthritis is collagen-induced arthritis. This model shares clinical, histological and immunological characteristics of human autoimmune rheumatoid arthritis and is an acceptable model for human autoimmune arthritis. Mouse and rat models are characterized by synovitis, erosion of cartilage and subchondral bone. The compounds of the invention can be tested for activity against autoimmune arthritis using the protocols described in Current Protocols in Immunology, above, units 15.5. See also the model using a monoclonal antibody to CD18 and VLA-4 integrins described in Issekutz, A. C. et al., Immunology, (1996) 88:569.

A model of asthma has been described in which antigen-induced airway hyper-reactivity, pulmonary eosinophilia and inflammation are induced by sensitizing an animal with ovalbumin and then challenging the animal with the same protein delivered by aerosol. Several animal models (guinea pig, rat, non-human primate) show symptoms similar to atopic asthma in humans upon challenge with aerosol antigens. Murine models have many of the features of human asthma. Suitable procedures to test the compositions of the invention for activity and effectiveness in the treatment of asthma are described by Wolyniec, W. W. et al., Am. J. Respir. Cell Mol. Biol., (1998) 18:777 and the references cited therein.

Additionally, the compositions of the invention can be tested on animal models for psoriasis like diseases. The compounds of the invention can be tested in the scid/scid mouse model described by Schon, M. P. et al., Nat. Med., (1997) 3:183, in which the mice demonstrate histopathologic skin lesions resembling psoriasis. Another suitable model is the human skin/scid mouse chimera prepared as described by Nickoloff, B. J. et al., Am. J. Path., (1995) 146:580.

Various animal models are well known for testing anti-cancer activity of a candidate therapeutic composition. These include human tumor xenografting into athymic nude mice or scid/scid mice, or genetic murine tumor models such as p53 knockout mice.

Recombinant (transgenic) animal models can be engineered by introducing the coding portion of the molecules identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g. baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA, 82, 6148-615 [1985]); gene targeting in embryonic stem cells (Thompson et al., Cell, 56, 313-321 [1989]); electroporation of embryos (Lo, Mol. Cel. Biol., 3, 1803-1814 [1983)); sperm-mediated gene transfer (Lavitrano et al., Cell, 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89, 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals may be further examined for signs of immune disease pathology, for example by histological examination to determine infiltration of immune cells into specific tissues or for the presence of cancerous or malignant tissue.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector (see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

### E. Formulations

The TACIs or BR3 molecules, or antagonists or agonists described herein, are optionally employed in a carrier. Suitable carriers and their formulations are described in Remington's Pharmaceutical Sciences, 16th ed., 1980, Mack Publishing Co., edited by Osol et al. Typically, an appropriate amount of a pharmaceutically-acceptable salt is used in the carrier to render the formulation isotonic. Examples of the carrier include saline, Ringer's solution and dextrose solution. The pH of the carrier is preferably from about 5 to about 8, and more preferably from about 7.4 to about 7.8. It will be apparent to those persons skilled in the art that certain carriers may be more preferable depending upon, for instance, the route of administration and concentration of active agent being administered. The carrier may be in the form of a lyophilized formulation or aqueous solution.

Acceptable carriers, excipients, or stabilizers are preferably nontoxic to cells and/or recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN™, PLURONICS™ or polyethylene glycol (PEG).

The formulation may also contain more than one active compound as necessary for the particular indication being treated, preferably those with complementary activities that do not adversely affect each other.

The TACIs or BR3, or antagonist or agonist described herein, may also be entrapped in inicrocapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for In vivo administration should be sterile. This is readily accomplished by filtration through sterile filtration membranes.

Sustained-release preparations may be prepared. Suitable examples of sustained-release preparations include semipermeable matrices of solid hydrophobic polymers containing the active agent, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

### F. Modes of Therapy

The molecules described herein are useful in treating various pathological conditions, such as immune related diseases or cancer. These conditions can be treated by stimulating or inhibiting a selected activity associated with TALL-1, APRIL, TACI, BCMA, TACIs or BR3 in a mammal through, for example, administration of one or more antagonists or agonists described herein.

Diagnosis in mammals of the various pathological conditions described herein can be made by the skilled practitioner. Diagnostic techniques are available in the art which allow, e.g., for the diagnosis or detection of cancer or immune related disease in a mammal. For instance, cancers may be identified through techniques, including but not limited to, palpation, blood analysis, x-ray, NMR and the like. Immune related diseases can also be readily identified. In systemic lupus erythematosus, the central mediator of disease is the production of auto-reactive antibodies to self proteins/tissues and the subsequent generation of immune-mediated inflammation. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may. mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjogren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barr syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are Infectious disease including but not limited to viral infection (including but not limited to AIDS, hepatitis A, B, C, D, E) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e. as from chemotherapy) immunodeficiency), and neoplasia.

The antagonist(s) or agonist(s) can be administered in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. Optionally, administration may be performed through mini-pump infusion using various commercially available devices. The antagonists or agonists may also be employed using gene therapy techniques which have been described in the art.

Effective dosages and schedules for administering antagonists or agonists may be determined empirically, and making such determinations is within the skill in the art. Single or multiple dosages may be employed. It is presently believed that an effective dosage or amount of antagonist or agonist used alone may range from about 1 μg/kg to about 100 mg/kg of body weight or more per day. Interspecies scaling of dosages can be performed in a manner known in the art, eg., as disclosed in Mordenti et al., Pharmaceut. Res., 8:1351 (1991).

When in vivo administration of an agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue. Those skilled in the art will understand that the dosage of antagonist or agonist that must be administered will vary depending on, for example, the mammal which will receive the agonist or antagonist, the route of administration, and other drugs or therapies being administered to the mammal.

Depending on the type of cells and/or severity of the disease, about 1 μg/kg to 15 mg/kg (e.g. 0.1-20 mg/kg) of antagonist antibody or agonist antibody is an initial candidate dosage for administration, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful.

Optionally, prior to administration of any antagonist or agonist, the mammal or patient can be tested to determine levels or activity of TALL-1, APRIL, TACI, BCMA, TACIs or BR3. Such testing may be conducted by ELISA or FACS of serum samples or peripheral blood leukocytes.

A single type of antagonist or agonist may be used in the methods of the invention. For example, a TALL-1 antagonist, such as a TACIs receptor immunoadhesin molecule, may be administered. Alternatively, the skilled practitioner may opt to employ a combination of antagonists or agonists in the methods, e.g., a combination of a TACIs receptor immunoadhesin and an anti-APRIL antibody. It may further be desirable to employ a dual antagonist, i.e., an antagonist which acts to block or inhibit both TALL-1 and APRIL. Such an antagonist molecule may, for instance, bind to epitopes conserved between TALL-1 and APRIL, or TACI, TACIs, BR3, and BCMA.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity in Section I above. In addition, therapies based on therapeutic antibodies that target tumor antigens such as Rituxan™ or Herceptin™ as well as anti-angiogenic antibodies such as anti-VEGF.

Preparation and dosing schedules for chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of, e.g. an antagonist, or may be given simultaneously therewith. The antagonist, for instance, may also be combined with an anti-oestrogen compound such as tamoxifen or an anti-progesterone such as onapristone (see, EP 616812) in dosages known for such molecules.

It may be desirable to also administer antibodies against other antigens, such as antibodies which bind to CD20, CD11a, CD18, CD40, ErbB2, EGFR, ErbB3, ErbB4, vascular endothelial factor (VEGF), or other TNFR family members (such as DR4, DR5, OPG, TNFR1, TNFR2). Alternatively, or in addition, two or more antibodies binding the same or two or more different antigens disclosed herein may be co-administered to the patient. Sometimes, it may be beneficial to also administer one or more cytokines to the patient. In one embodiment, the antagonists herein are co-administered with a growth inhibitory agent. For example, the growth inhibitory agent may be administered first, followed by an antagonist of the present invention.

The antagonist or agonist (and one or more other therapies) may be administered concurrently or sequentially. Following administration of antagonist or agonist, treated cells in vitro can be analyzed. Where there has been in vivo treatment, a treated mammal can be monitored in various ways well known to the skilled practitioner. For instance, markers of B cell activity such as Ig production (non-specific or antigen specific) can be assayed.

### G. Methods of Screening

The invention also encompasses methods of screening molecules to identify those which can act as agonists or antagonists of the APRIL/TACIs interaction or the TALL-1/TACIs/BR3 interaction. Such molecules may comprise small molecules or polypeptides, including antibodies. Examples of small molecules include, but are not limited to, small peptides or peptide-like molecules, preferably soluble peptides, and synthetic non-peptidyl organic or inorganic compounds. The screening assays for drug candidates are designed to identify compounds or molecules that bind or complex with the ligand or receptor polypeptides identified herein, or otherwise interfere with the interaction of these polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

Assays for, for instance, antagonists are common in that they call for contacting the drug candidate with a ligand or receptor polypeptide identified herein under conditions and for a time sufficient to allow these two components to interact.

In binding assays, the interaction is binding and the complex formed can be isolated-or detected in the reaction mixture. In a particular embodiment, the ligand or receptor polypeptide identified herein or the drug candidate is immobilized on a solid phase, e.g., on a microtiter plate, by covalent or non-covalent attachments. Non-covalent attachment generally is accomplished by coating the solid surface with a solution of the ligand or receptor polypeptide and drying. Alternatively, an immobilized antibody, e.g., a monoclonal antibody, specific for the ligand or receptor polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular ligand or receptor polypeptide identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds or molecules that interfere with the interaction of a ligand or receptor polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the ligand or receptor polypeptide may be added to a cell along with the compound to be screened for a particular activity and the ability of the compound to inhibit the activity of interest in the presence of the ligand or receptor polypeptide indicates that the compound is an antagonist to the ligand or receptor polypeptide. Alternatively, antagonists may be detected by combining the ligand or receptor polypeptide and a potential antagonist with membrane-bound polypeptide receptors or recombinant receptors under appropriate conditions for a competitive inhibition assay. The ligand or receptor polypeptide can be labeled, such as by radioactivity, such that the number of polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the ligand or receptor polypeptide and a cDNA library created from this RNA is divided into pools and used to transfect COS cells or other cells that are not responsive to the ligand or receptor polypeptide. Transfected cells that are grown on glass slides are exposed to labeled ligand or receptor polypeptide. The ligand or receptor polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach, labeled ligand polypeptide can be photoaffinity-linked with cell membrane or extract preparations that express receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

### H. Articles of Manufacture

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agents in the composition may comprise antagonist(s) or agonist(s). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following examples are offered by way of illustration and not by way of limitation. The disclosures of all citations in the specification are expressly incorporated herein by reference.

### EXAMPLE 1

### Identification and Expression Cloning of TACIs and BR3

A chimeric protein, referred to as “AP-TALL-1”, was prepared using human placenta alkaline phosphatase (AP) fused to the N-terminus of a TALL-1 polypeptide consisting of amino acids 136-285 shown in FIG. 3. The AP was obtained by PCR amplification using pAPtag-5 (Genehunter Corporation) as a template, and fused and cloned into the expression vector, pCMV-1 Flag (Sigma), with AP at the N-terminus of TALL-1. The AP-TALL-1 was transiently transfected (using Lipofectamine reagent; Gibco-BRL) and expressed in human embryonic kidney 293 cells (ATCC). The conditioned medium from the transfected 293 cells was filtered (0.45 micron), stored at 4° C. in a buffer containing 20 MM Hepes (pH 7.0) and 1 mM sodium azide, and used for subsequent cell staining procedures.

To identify a receptor for TALL-1, a cDNA expression library was constructed in pRK5 vector (EP 307,247, published Mar. 15, 1989) using PolyA+ mRNA derived from human spleen and IM-9 cells [Flanagan et al., Cell, 63:185 (1990); Tartaglia et al., Cell, 83:1263-1271 (1995)]. Pools of ˜1000 cDNA clones (Miniprep DNA (Qiagen)) from the library were transfected (using Lipofectamine) into COS 7 cells (ATCC) in 12 well plates using Fugene 6 (Roche Molecular Biochemicals), which after 36-48 hours, were then incubated with AP-TALL-1 conditioned medium, washed, and stained for AP activity in situ. [Yan et al., supra (2000)]. A positive pool was broken down to successively smaller size pools which contained neither TACI nor BCMA. After rounds of screening, cDNA encoding an AP-TALL-1 binding activity was identified. Sequencing of the cDNA insert revealed a single open reading frame predicted to encode a protein with a single predicted transmembrane region. This polypeptide (amino acid residues 1-184 of FIG. 6B) was referred to as BR3. (Another cDNA encoding an AP-TALL-1 and AP-APRIL binding activity was also identified, and this molecule is identified as TACIs, described further below).

Sequence alignments indicated that the BR3 molecule was likely not a member of the TNF-receptor superfamily, which superfamily is typically defined by the presence of characteristic, multiple cysteine-rich repeats within the extracellular ligand binding domain. These amino acid pseudorepeats are typically defined by 3 intramolecular disulfide bridges formed by 6 highly conserved cysteines [Locksley et al., Cell, 104:487-501 (2001)]. Furthermore, the extracellular domain of BR3 showed no homology to any member of the TNF-receptor family. In addition, the BR3 contained only four cysteine residues in its ectodomain. Database searches revealed a putative murine orthologue of BR3 (GenBank accession number AK008142). Similar to human BR3 identified above, the murine BR3 (mBR3) possessed only four cysteine residues. Overall, the hBR3 and mBR3 exhibited 56% identity. Both the hBR3 and mBR3 lacked an NH₂-terminal signal peptide, indicating that they are type III transmembrane proteins [Wilson-Rawls et al., Virology, 201:66-76 (1994)]. The intracellular domain of BR3 appeared to be highly conserved between hBR3 and mBR3.

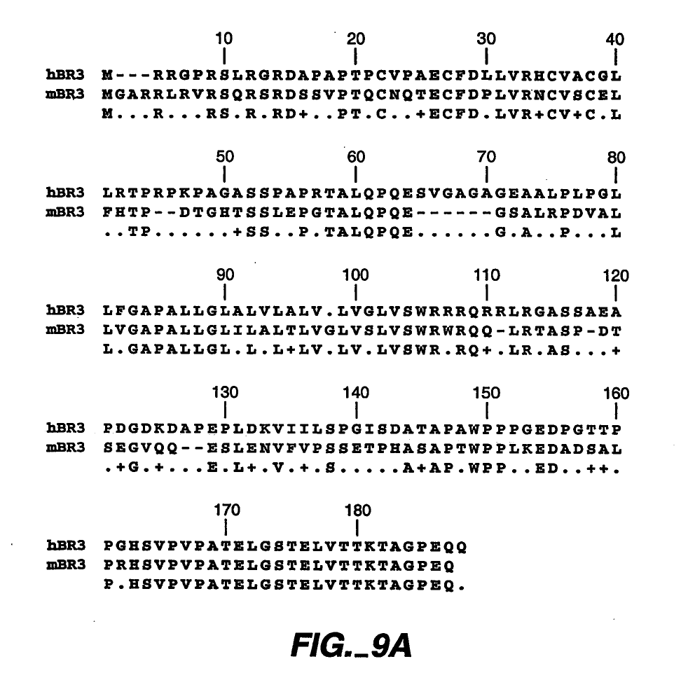

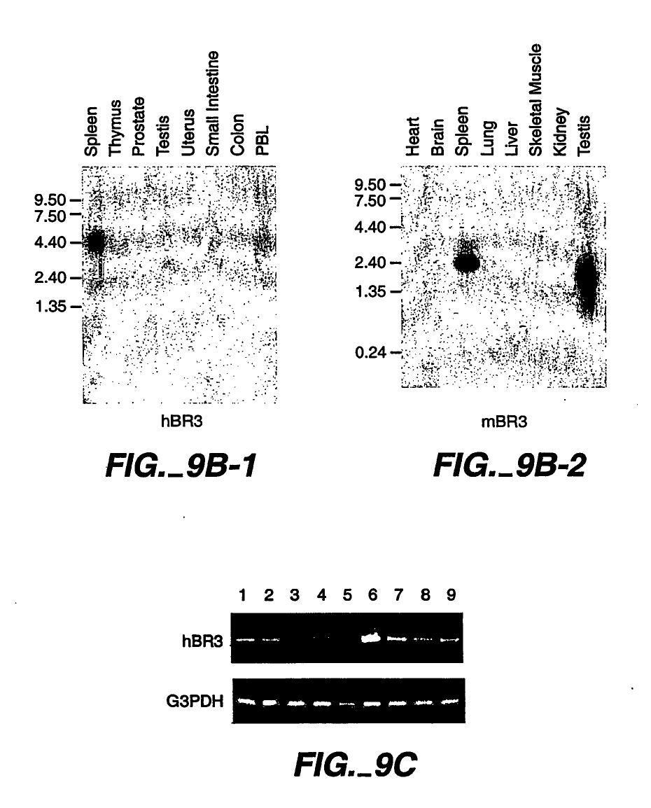

Northern Blotting was conducted according to common procedures known to those of skill in the art. Briefly, human and murine polyA+ RNA normal tissue blots (Clontech) were hybridized according to the manufacturer's instructions. ³²P-labeled probes were generated using DNA fragments corresponding to the nucleotide coding region of human or murine BR3. As shown in FIG. 9B, relatively high expression levels were detected in human and murine spleen tissue and in murine testis.

Furthermore, PCR analysis of a human cDNA panel showed highest expession of human BR3 in resting CD19+ B-cells (FIG. 9C), consistent with BR3 being a receptor for TALL-1 or B-cells. The expression pattern of human BR3 is thus distinct from that of TACI and BCMA. While BCMA appears to be B cell specific and TACI is expressed by both B cells and activated T cells [Laabi et al., Science, 289:883-884 (2000); Gras et al., Int. Immunol., 7:1093-1106 (1995); von Bulow et al., Science, 278:138-141 (1997); Khare et al., Trends Immunol., 22:61-63 (2001)], BR³ is highly expressed by resting B cells and is also detectable in resting T cells. The gene for murine BR3 was reported to be transcriptionally activated in one of four AKXD mouse strains susceptible to B-cell leukemia and lymphoma [Hansen et al., Genome Res. 10:237-243 (2000)].

Flag-tagged ligands were prepared as follows. Amino acids 105-250 of APRIL (see FIG. 4) were cloned into pCMV-1 Flag (Sigma), at HindIII site, resulting in fusion to amino acids 1-24 of the Flag signal and tag sequence. Amino acids 124-285 of TALL-1 (see FIG. 3) were fused to amino acids 1-27 of the Flag signal and tag sequence, as described above for Flag-APRIL, except that the NotI site was used. AP-APRIL was prepared by cloning amino acids 105-250 of APRIL (see FIG. 4) into a pCMV-1 Flag vector encoding human placental alkaline phosphatase such that the APRIL encoding sequence was fused C-terminally to AP, while the AP was fused C-terminally to Flag. AP-TALL-1 was prepared by cloning amino acids 136-285 of TALL-1 (see FIG. 3) into the pCMV-1 Flag, AP vector, as described above for AP-APRIL. The respective tagged proteins were then expressed in 293 cells or CHO cells and purified using M2 anti-Flag resin (Sigma).

One μg of the purified Flag-APRIL or Flag-TALL-1 was incubated with 1 μg of purified human immunoadhesin containing the IgG1-Fc fusion of the ECD of BR3 or TACI overnight at 4° C. The TACI-ECD.hFc immunoadhesins were prepared by methods described in Ashkenazi et al., Proc. Natl. Acad. Sci., 88:10535-10539 (1991). The immunoadhesin constructs consisted of amino acids 2-166 of the human TACI polypeptide (see FIG. 1). The TACI-ECD constructs were expressed in CHO cells using a heterologous signal sequence (pre-pro trypsin amino acids 1-17 of pCMV-1 Flag (Sigma)) and encoding the human IgG1 Fc region downstream of the TACI sequence, and then purified by protein A affinity chromatography. The BR3-ECD immunoadhesins were prepared by methods described in Ashkenazi et al., as cited above. The immunoadhesin constructs consisted of amino acids 2-62 of the human BR3 polypeptide (see FIG. 6B). The BR3-ECD constructs were expressed in CHO cells using a heterologous signal sequence (pre-pro trypsin amino acids 1-17 of pCMV-1 Flag (Sigma)) and encoding the human IgG1 Fc region downstream of the BCMA sequence, and then purified by protein A affinity chromatography.

The mixture was subjected to immunoprecipitation through the receptor-immunoadhesin with protein A-agarose (Repligen). The immunoprecipitates were then analyzed by Western blot with horseradish peroxidase-conjugated anti-Flag M2 mAb (Sigma) to detect the Flag-tagged ligands. Flag-TALL-1, but not Flag-APRIL, was readily detected in complex with hBR3-hFc whereas TACI-hFc bound both Flag-TALL-1 and Flag-APRIL. These results show that, unlike TACI and BCMA, BR3 specifically binds TALL-1 but not APRIL.

In an in vitro assay, COS 7 cells (ATCC) were seeded into 12 well plates 24 hours before transfection. The cells were then transfected with 1 microgram TACI (the 265 amino acid form of human TACI described above, cloned in pRK5B vector, infra) or vector plasmid (pRK5B) alone. 18-24 hours after transfection, the cells were incubated with conditioned medium containing AP-TALL-1 or AP-APRIL for 1 hour at room temperature and stained for AP activity in situ as described in Tartaglia et al., Cell, 83:1263-1271 (1995).

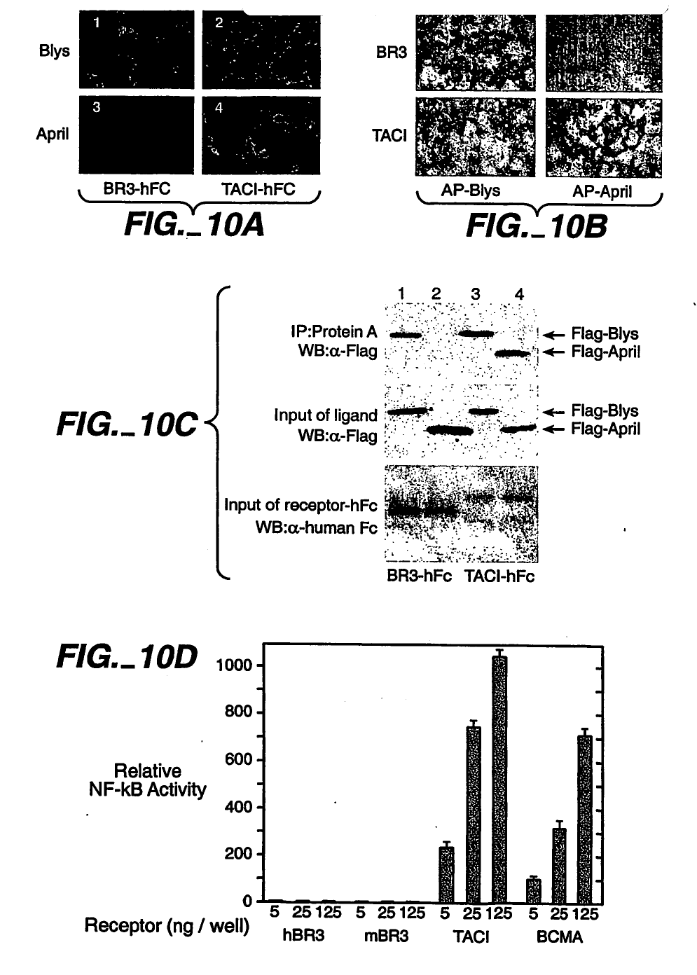

Transfection of a hBR3 or mBR3 expression construct into COS 7 cells conferred strong binding to AP-TALL-1, but not to AP-APRIL (FIG. 10 and data not shown). In contrast, both AP-TALL-1 and AP-APRIL bound to TACI-transfected cells. A human Fc fusion protein containing the ectodomain of hBR3(hBR3-hFc) bound to COS 7 cells transfected with an expression construct encoding the full-length transmembrane form of TALL-1, but not to cells expressing APRIL.

Human TNF-alpha was cloned into pRK5B vector (pRK5B is a precursor of pRK5D that does not contain the SfiI site; see Holmes et al., Science, 253:1278-1280 (1991)). For the detection of TNF-alpha expression on the cell surface, a Flag tag was inserted between amino acid 70 and amino acid 71 (using the numbering according to the sequence in Pennica et al., supra). An extracellular region of TALL-1 (aa 75-285; see FIG. 3), 4-1BBL (aa 59-254; Goodwin et al., Eur. J. Immunol., 23:2631-2641 (1993)), CD27 ligand (aa 40-193; Goodwin et al., Cell, 73:447-456 (1993)), CD30 ligand (aa 61-234; Smith et al., Cell, 73:1349-1360 (1993)), RANKL (aa 71-317; see WO98/28426), Apo-2 ligald (aa 40-281; see WO97/25428) or Apo-3L (aa 46-249; see WO99/19490) was individually cloned at the BamHI site. This resulted in a chimeric ligand with the intracellular and transmembrane regions from TNF-alpha and the extracellular region from the various ligands. For APRIL (see FIG. 4) and EDA-A1, EDA-A2 (Srivastava et al., supra), full length cDNA clones without Flag tag were used.

Transfected COS 7 cells were subsequently incubated with TACI.ECD.hFC immunoadhesin, hBr3-hFc, or mBR3-hFc (prepared as described above). Cells were incubated with the TACI ECD-IgG (or a TNFR1-IgG construct prepared as described in Ashkenazi et al., Proc. Natl. Acad. Sci., 88:10535-10539 (1991)) at 1 μg/ml for 1 hour in PBS. Cells were subsequently washed three times with PBS and fixed with 4% paraformaldehyde in PBS. Cell staining was visualized by incubation with biotinylated goat anti-human antibody (Jackson Labs, at 1:200 dilution) followed by Cy3-streptavidin (Jackson Labs, at 1:200 dilution). Murine BR3-Fc, like hBR3-hFc, also only bound TALL-1-transfected but not APRIL-transfected COS 7 cells (data not shown). Further, hBR3-hFc failed to bind to cells expressing several other TNF family members, including CD27L, CD30L, CD40L, EDA-A1, EDA-A2, 4-1BBL, FasL, Apo2L/TRAIL, Apo3L/TWEAK, OX-40L, RANKL/TRANCE, or GITRL (data not shown). In contrast, TACI-hFc fusion protein bound cells transfected with either TALL-1 or APRIL (FIG. 10).

In an NF-kB assay, 293 cells (ATCC) were seeded 24 hours before transfection at 1×10⁵ cells/well into 12-well plates and transfected with 0.25 μg of ELAM-luciferase reporter gene plasmid, 25 ng pRL-TK (Promega) and the indicated amounts of each expression construct (see FIG. 10). Total amount of transfected DNA was kept constant at 1 mg by supplementation with empty pRK5B vector. Cells were harvested 20-24 hours after transfection and reporter gene activity determined with the Dual-Luciferase Reporter Assay System (Promega).

Upon transfection into 293 cells, both TACI and BCMA induced profound NF-kB activation in a does dependent manner, as determined by the reporter gene assay. Under similar conditions, neither hBR3 nor mBR3 triggered detectable activation of NF-kB (FIG. 10). The failure of BR3 to activate NF-kB in this assay was unlikely due to poor expression of BR3 since BR3-transfected cells bound ligand (AP-TALL-1) at a level equivalent to TACI or BCMA-transfected cells (FIG. 10 and data not shown).

While the prominent expression of BR3 in the spleen and in. particular, by B cells, is consistent with it being a functional receptor for TALL-1, its expression pattern is distinct from that of TACI and BCMA. BCMA is B cell specific and TACI is expressed by both B-cells and activated T-cells [Laabi et al., Science, 289:883-884 (2000); Gras et al., Int. Immunol., 7:1093-1106 (1995); von Bulow et al., Science, 278:138-141 (1997); Khare et al., Trends Immunol., 22:61-63 (2001)]. In contrast, BR3 is highly expressed by resting B cells and detectable in resting T cells. It appears to be downregulated upon activation. Interestingly, the gene for mBR3 was reported to be transcriptionally activated in one of four AKXD mouse strains susceptible to B-cell leukemia and lymphoma [Hansen et al., Genome Res., 10:237-243 (2000)]. It is believed that BR3 may be an important receptor in B-cell homeostasis and its dysregulation may contribute to the development of B-cell neoplasms.

Both APRIL and TALL-1 bind to TACI and BCMA; however, some preference in binding is observed with TACI-TALL-1 and BCMA-APRIL being preferred partners [Marsters et al., supra (2000)]. In contrast, the experiments herein showed BR3 bound to TALL-1 but not to APRIL. Although APRIL, originally identified as a tumor cell growth factor, has been shown to bind both TACI and BCMA [Marsters et al., supra (2000) ; Wu et al., J. Biol. Chem., 275 :35478 (2000); Yu et al., Nat. Immunol., 1 :252-256 (2000)], its physiological role(s) in B cell function is not fully understood. Since BR3 is specific for TALL-1, it is believed that administration of BR3-Fc (such as administration of the immunoadhesin to mice) should block TALL-1 but not APRIL induced activation of TACI and BCMA.

A study of B cell deficient A/WySnJ mice, that unlike the related A/J strain possess a single autosomal codominant locus termed Bcmd (for B cell maturation defect) that is responsible for the profound deficit in peripheral B cells, is described in Lentz et al., J. Immunol., 157:598-606 (1996); Lentz et al., J. Immunol., 160:3743-3747 (1998); Hoag et al., Immunogenetics, 51:924-929 (2000)]. The Bcmd locus maps to the middle region of mouse chromosome 15 which is syntenic to where BR3 maps on human chromosome 22. Splenic B cells from the A/WySnJ mice are reported to not exhibit a proliferative response to recombinant TALL-1 either in vitro or in vivo. It is presently believed that the gene defect in such A/WySnJ mice, as defined genetically by the Bcmd locus, is in the gene encoding BR3. In Applicants' experiments, RT-PCR analysis failed to reveal the presence of BR3 transcript in splenic or B cell RNA from A/WySnJ mice (obtained from Dr. Michael Cancro, University of Pennsylvania, Philadelphia, Pa.) while the transcript for TACI control was easily detectable in the same samples. In contrast, the complete BR3 coding gene was easily detectable in A/J mice. These data are consistent with inactivation of BR3 by gene deletion as being responsible for the lack of peripheral B cells observed in A/WySnJ mice. It is presently believed that the signaling pathway engaged by BR3 may be responsible for the B cell proliferative effects of TALL-1 and that in the absence of BR3, B cell homeostasis may be compromised.

The TACIs molecule, referred to above, identified in the screening was found to encode a polypeptide comprising the amino acids 1 to 246 of FIG. 5B. Like BR3, the polypeptide appears to include a single cysteine-rich domain. The putative ECD comprises amino acid residues 1 to 119 of FIG. 5B. In in vitro binding assays (performed as described above to detect AP-TALL-1 staining and AP-APRIL staining), it was found that TACIs binds to both TALL-1 and APRIL (data not shown).

### EXAMPLE 2

### Expression of TACIs Polypeptides or BR3 Polypeptides in E. coli

This example illustrates the preparation of forms of TACIs polypeptides and forms of BR3 polypeptides by recombinant expression in E. coli.

For expression of TACIs polypeptide, the DNA sequence encoding the full-length TACIs polypeptide or a fragment or variant thereof is initially amplified using selected PCR primers. For expression of BR3 polypeptide, the DNA sequence encoding the full-length BR3 polypeptide or a fragment or variant thereof is initially amplified using selected PCR primers.

The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the TACIs polypeptide coding region or the BR3 polypeptide coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized TACIs polypeptide or the solubilized BR3 polypeptide can then be purified using a metal chelating column under conditions that allow tight binding of the polypeptide.

### EXAMPLE 3

### Expression of TACIs Polypeptides or BR3 Polypeptides in Mammalian Cells

This example illustrates preparation of forms of TACIs polypeptides and BR3 polypeptides by recombinant expression in mammalian cells.

The vector, pRK5 (see EP 307,247, published Mar. 15, 1989), is employed as the expression vector. Optionally, the TACIs polypeptide-encoding DNA is ligated into pRK5 with selected restriction enzymes to allow insertion of the TACIs polypeptide-encoding DNA using ligation methods such as described in Sambrook et al., supra. The resulting vector is called pRK5-TACIs polypeptide. Optionally, the BR3 polypeptide-encoding DNA is ligated into pRK5 with selected restriction enzymes to allow insertion of the BR3 polypeptide-encoding DNA using ligation methods such as described in Sambrook et al., supra. The resulting vector is called pRK5-BR3 polypeptide.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 microgram pRK5-TACIs polypeptide DNA is mixed with about 1 microgram DNA encoding the VA RNA gene [Thimmappaya et al., Cell, 31:543 (1982)] and dissolved in 500 microliter of 1 mM Tris-HCl, 0.1 nM EDTA, 0.227 M CaCl₂. Alternatively, about 10 microgram pRK5-BR3 polypeptide DNA is mixed with about 1 microgram DNA encoding the VA RNA gene [Thimmappaya et al., Cell, 31:543 (1982)] and dissolved in 500 microliter of 1 mM Tris-HCl, 0.1 mM EDTA, 0.227 M CaCl₂. To the vector mixture is added, dropwise, 500 microliter of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 microCi/ml ³⁵S-cysteine and 200 microci/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of TACIs polypeptide or the presence of BR3 polypeptide. The cultures containing transfected cells may undergo further incubation (in serum free medium) and the medium is tested in selected bioassays.

In an alternative technique, TACIs polypeptide-encoding DNA or BR3 polypeptide-encoding DNA may be introduced into 293 cells transiently using the dextran sulfate method described by Somparyrac et al., Proc. Natl. Acad. Sci., 12:7575 (1981). 293 cells are grown to maximal density in a spinner flask and followed by addition of 700 microgram pRK5-TACIs polypeptide DNA, or by addition of 700 microgram BR3 polypeptide DNA. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 microgram/ml bovine insulin and 0.1 microgram/ml bovine transferrin. After about four days, the conditioned media is centrifuged and filtered to remove cells and debris. The sample containing expressed TACIs polypeptide or expressed BR3 polypeptide can then be concentrated and purified by any selected method, such as dialysis and/or column chromatography.

In another embodiment, TACIs polypeptide or BR3 polypeptide can be expressed in CHO cells. The pRK5-TACIs polypeptide vector or the pRK5-BR3 polypeptide vector can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of the desired polypeptide, the culture medium may be replaced with serum free medium. Preferably, the cultures are incubated for about 6 days, and then the conditioned medium is harvested. The medium containing the expressed TACIs polypeptide or BR3 polypeptide can then be concentrated and purified by any selected method.

Epitope-tagged TACIs polypeptide or epitope-tagged BR3 polypeptide may also be expressed in host CHO cells. The TACIs polypeptide-encoding DNA or the BR3 polypeptide-encoding DNA may be subcloned out of the pRK5 vector. The subclone insert can undergo PCR to fuse in frame with a selected epitope tag such as a poly-his tag into a Baculovirus expression vector. The poly-his tagged TACIs polypeptide-encoding DNA insert or the poly-his tagged BR3 polypeptide-encoding DNA insert can then be subcloned into an SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged TACIs polypeptide or the expressed poly-His tagged BR3 polypeptide can then be concentrated and purified by any selected method, such as by Ni²⁺-chelate affinity chromatography.

### EXAMPLE 4

### Expression of a TACIs Polypeptide or a BR3 Polypeptide in Yeast

The following method describes recombinant expression of TACIs polypeptides and BR3 polypeptides in yeast.

First, yeast expression vectors are constructed for intracellular production or secretion of TACIs polypeptide from the ADH2/GAPDH promoter. DNA encoding the TACIs polypeptide of interest, a selected signal peptide and the promoter is inserted into suitable restriction enzyme sites in the selected plasmid to direct intracellular expression of the TACIs polypeptide. For secretion, DNA encoding the TACIs polypeptide can be cloned into the selected plasmid, together with DNA encoding the ADH2/GAPDH promoter, the yeast alpha-factor secretory signal/leader sequence, and linker sequences (if needed) for expression of the TACIs polypeptide.

Alternatively, yeast expression vectors are constructed for intracellular production or secretion of BR3 polypeptide from the ADH2/GAPDH promoter. DNA encoding the BR3 polypeptide of interest, a selected signal peptide and the promoter is inserted into suitable restriction enzyme sites in the selected plasmid to direct intracellular expression of the BR3 polypeptide. For secretion, DNA encoding the BR3 polypeptide can be cloned into the selected plasmid, together with DNA encoding the ADH2/GAPDH promoter, the yeast alpha-factor secretory signal/leader sequence, and linker sequences (if needed) for expression of the BR3 polypeptide.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant TACIs polypeptide or BR3 polypeptide can subsequently be isolated and purified by removing the yeast cells from the fermentation medium by centrifugation and then concentrating the medium using selected cartridge filters. The concentrate containing the TACIs polypeptide or BR3 polypeptide may further be purified using selected column chromatography resins.

### EXAMPLE 5

### Expression of TACIs Polypeptide or BR3 Polypeptides in Baculovirus-Infected Insect Cells

The following method describes recombinant expression of TACIs polypeptides and BR3 polypeptides in Baculovirus-infected insect cells.

The TACIs polypeptide-encoding DNA or the BR3 polypeptide-encoding DNA is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the TACIs polypeptide-encoding DNA or the desired portion of the TACIs polypeptide-encoding DNA (such as the sequence encoding the extracellular domain of a transmembrane protein) is amplified by PCR with primers complementary to the 5′ and 3′ regions. Alternatively, the BR3 polypeptide-encoding DNA or the desired portion of the BR3 polypeptide-encoding DNA (such as the sequence encoding the extracellular domain of a transmembrane protein) is amplified by PCR with primers complementary to the 5′ and 3′ regions. The 5′ primer may incorporate flanking (selected) restriction enzyme sites. The product is then digested with those selected restriction enzymes and subdloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4 to 5 days of incubation at 28° C., the released viruses are harvested and used for further amplifications. Viral infection and protein expression is performed as described by O'Reilley et al., Baculovirus expression vectors: A laboratory Manual, Oxford:Oxford University Press (1994).

Expressed poly-his tagged TACIs polypeptide or expressed poly-his tagged BR3 polypeptide can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% Glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% Glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% Glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or western blot with Ni ²⁺-NTA-conjugated to alkaline phosphatase (Qiagen). Fractions containing the eluted His₁₀-tagged TACIs polypeptide or the eluted His₁₀-tagged BR3 polypeptide are pooled and dialyzed against loading buffer.

Alternatively, purification of the IgG tagged (or Fc tagged) TACIs polypeptide or the IgG tagged (or Fc tagged) BR3 polypeptide can be performed using known chromatography techniques, including for instance, Protein A or protein G column chromatography.

### EXAMPLE 6

### Preparation of Antibodies that Bind TACIs Polypeptides and/or BR3 Polypeptides

This example illustrates the preparation of monoclonal antibodies which can specifically bind to TACIs polypeptides and/or BR3 polypeptides.

Techniques for producing the monoclonal antibodies are known in the art and are described, for instance, in Goding, supra. Immunogens that may be employed include purified TACIs polypeptide, purified BR3 polypeptide, fusion proteins containing a TACIs polypeptide, fusion proteins containing a BR3 polypeptide, cells expressing recombinant TACIs polypeptide on the cell surface, and cells expressing recombinant BR3 polypeptide on the cell surface. Selection of the immunogen can be made by the skilled artisan without undue experimentation.

Mice, such as Balb/c, are immunized with the TACIs polypeptide immunogen, or BR3 polypeptide immunogen, emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-TACIs polypeptide antibodies or BR3 polypeptide antibodies.

After a suitable antibody titer has been detected, the animals “positive” for antibodies can be injected with a final intravenous injection of TACIs polypeptide or of BR3 polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against TACIs polypeptide or for reactivity against BR3 polypeptide. Determination of “positive” hybridoma cells secreting the desired monoclonal antibodies against a TACIs polypeptide or a BR3 polypeptide is within the skill in the art.

The positive hybridoma cells can be injected intraperitoneally into syngeneic Balb/c mice to produce ascites containing the anti-TACIs polypeptide monoclonal antibodies or anti-BR3 polypeptide monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### EXAMPLE 7

### Effects of BR3-Fc Polypeptides in In Vivo Lupus Model

The effects of BR3-Fc immunoadhesin polypeptides were examined in an in vivo murine model for systemic lupus erythematosus (SLE or lupus).

Murine BR3-Fc (immunoadhesin prepared as described in Example 1) was injected intraperitoneally into 6-month old NZB×NZW (F1) mice (12 mice per group) for a period of 5 weeks (three times per week at a dosage of 100 μg protein). The NZB×NZW (F1) mice were obtained from Jackson Labs and are an established animal model for early stage lupus (see, Relevance of systemic lupus erythematosus nephritis animal models to human disease, Foster M H, Sem. Nephrol., 19:12-24 (January 1999)). Control animals were similarly injected with saline.

The animals were examined bi-weekly for the following: survival analysis; body weight; dsDNA antibody titer; and proteinurea measurement. Proteinurea levels were measured in freshly collected urine samples from the treated and control animals using Multistix™ reagent strips (Bayer). DsDNA antibody levels were measured in the treated and untreated animals as follows. Serum was collected at 6, 8, and 9 months of age and tested in assay plates in accordance with the following protocol. 96-well plates (Nalgene NUNC-Immuno™ MaxiSorp™ surface plates) were coated with 100 μl of 50 μg poly-L-lysine (0.01% solution, Sigma) (diluted in 0.1M Tris, pH7.5) for four hours at room temperature. The plates were washed with 200 μl phosphate buffered saline (PBS) supplemented with 10% heat-inactivated fetal bovine serum (Gibco) three times. The plates were then coated with 100 μl of 20 μg/ml poly Deoxyadenylic-thymidylic acid (Sigma) (diluted in 0.1M Tris, pH7.5) overnight at 4° C. The plates were washed with 200 μl PBS supplemented with 10% fetal bovine serum (Gibco) three times. The plates were subsequently blocked with 100 μl PBS supplemented with 10% heat-inactivated fetal bovine serum (Gibco) for 1 hour at room temperature. The plates were washed with 200 μl PBS supplemented with 10% heat-inactivated fetal bovine serum (Gibco) three times. The plates were then incubated with 100 μl serum samples diluted 1:100 in PBS supplemented with 10% heat-inactivated fetal bovine serum for 2 hours at room temperature. The plates were washed with 200 μl PBS supplemented with 10% heat-inactivated fetal bovine serum three times. The plates were then incubated with 100 μl HRP-conjugated goat anti-mouse IgG1 (Caltag Labs) diluted 1:2000 in PBS supplemented with 10% heat-inactivated fetal bovine serum for 1 hour at room temperature. The plates were washed with 200 μl PBS supplemented with 10% heat-inactivated fetal bovine serum five times. The plates were then incubated with 100 μl developing agent (1:1 mixture of substrate reagents A and B, BD Pharmingen) for t0 minutes at room temperature. The reaction was stopped using 50 μl of 4.5 N sulphuric acid, and absorbance vaues were measured at 450 nm using a Spectramax 340 plate reader.

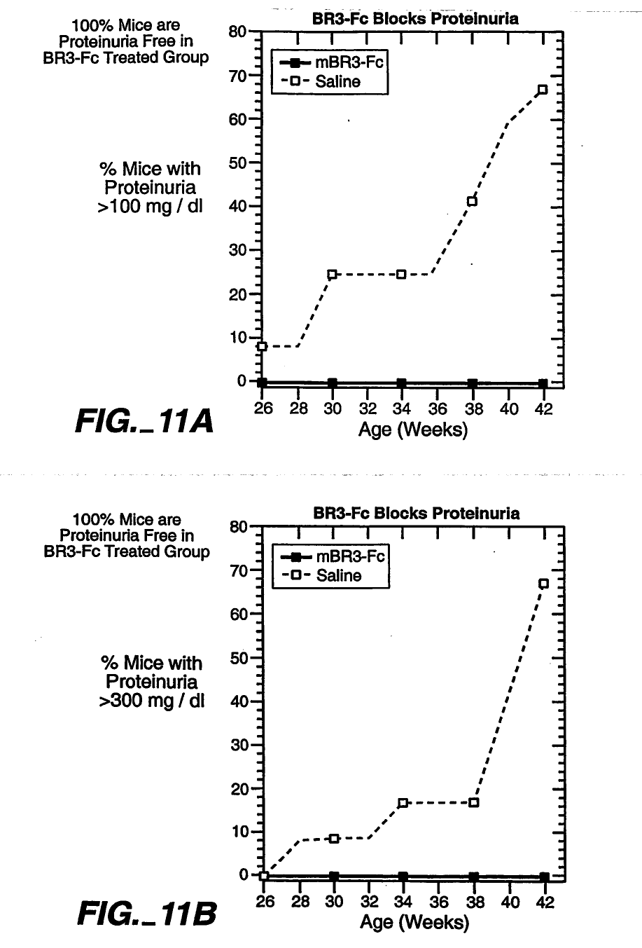

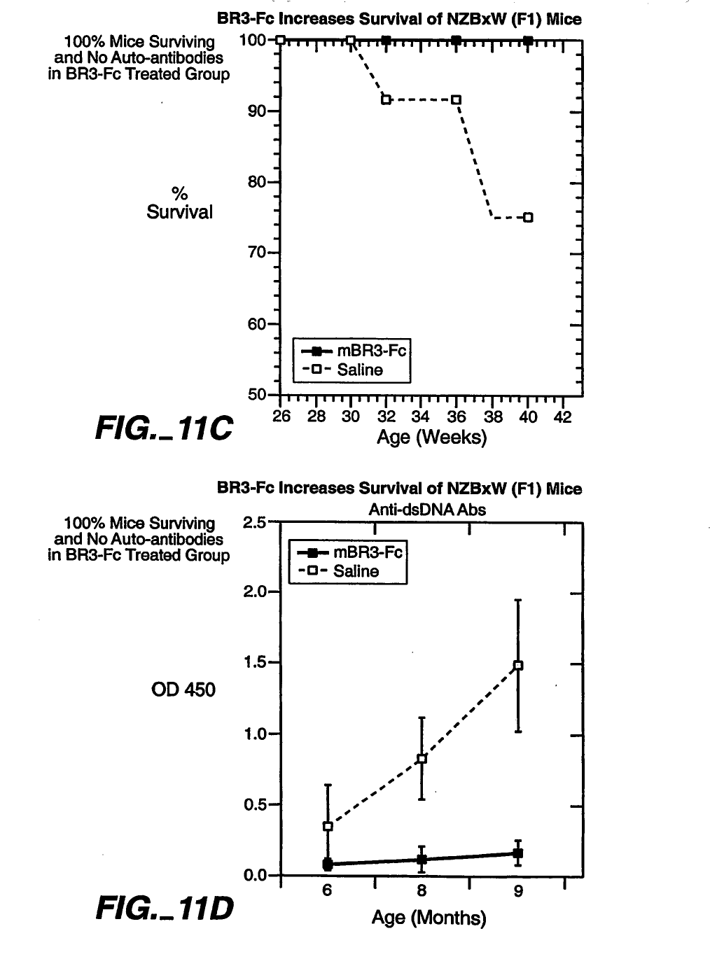

The results are shown in FIGS. 11A-11D. FIGS. 11A and 11B shows that the BR3-Fc treated animals were proteinurea free. In the NZB×NZW (F1) mouse, the typical (untreated) animal at 6 months of age has proteinurea levels of about 0-30 mg/dl, whereas at 12 months of age, the animals typically exhibit proteinurea levels >300 mg/dl, resulting in about 80% death. The data suggests that in the BR3-Fc treated animals, BR3-Fc is capable of blocking proteinurea during the course of lupus and protects against kidney damage.

The BR3-Fc treated animals also exhibited enhanced survival. As shown in FIG. 11C, administration of BR3-Fc enhanced survival of the animals. While survival was down to 75% in the control group at 40 weeks of age, 100% of the animals in the BR3-Fc treated group were alive. Levels of anti-dsDNA antibodies were also significantly lower in the BR3-Fc treated animals as compared to the control group at 9 months of age (FIG. 11D). These data suggest that BR3-Fc treatment blocked production of auto-antibodies by B cells in the lupus mice and enhanced survival by blocking TALL-1 function in vivo.

1. An isolated nucleic acid comprising (a) DNA encoding a TACIs polypeptide comprising the sequence of amino acid residues 1 to 246 of SEQ ID NO:14, or (b) the complement of the DNA molecule of (a).

2. The nucleic acid of claim 1, wherein said DNA comprises the coding nucleotide sequence of SEQ ID NO:13.

3. The nucleic acid of claim 1, wherein said DNA consists of the coding nucleotide sequence of SEQ ID NO:13.

4. An isolated nucleic acid comprising DNA which has (a) at least 95% sequence identity to the coding sequence of nucleotides of SEQ ID NO:13 and. (b) encodes aTACIs polypeptide.

5. An isolated nucleic acid comprising DNA from the group consisting of:
a) a DNA havinq at least 90% sequence identity to a DNA sequence encoding a TACIs polypeptide comprising amnio acid residues 1 to 246 of SEQ ID NO:14;
b) a DNA sequence that hybridizes under stringent conditions to a DNA of a);
c) a DNA sequence that, due to the degeneracy of the genetic code, encodes a TACIs polypeptide of a); and
d) DNA fully complementary to the DNA of a), b), or c).

6. A vector comprising the nucleic acid of claim 5.

7. The vector of claim 6 operably linked to control sequences recognized by a host cell transformed with the vector.

8. A host cell which includes the vector of claim 6.

9. The host cell of claim 8, wherein said cell is a CHO cell.

10. The host cell of claim 8, wherein said cell is an E.coli.

11. The host cell of claim 8, wherein said cell is a yeast cell.

12. A process for producing a TACIs polypeptide comprising culturing the host cell of claim 8 under conditions suitable for expression of said TACIs polypeptide and recovering said TACIs polypeptide from the cell culture.

13. An isolated TACIs polypeptide comprising amino acid residues 1 to 246 of FIG. 5B (SEQ ED NO:14).

14. An isolated TACIs polypeptide comprising the sequence of contiguous amino acid residues 1 to 246 of FIG. 5B (SEQ ID NO:14).

15. An isolated soluble TACIs polypeptide comprising amino acid residues 1 to 119 of FIG. 5B (SEQ ID NO:14).

16. An isolated TACIs polypeptide comprising a polypeptide selected from the group consisting of:
a) a TACIs polypeptide comprising amino acid residues 1 to 246 or 1 to 119 of FIG. 5B (SEQ ID NO:14) and
b) a fragment of a), wherein said fragment is a biologically active polypeptide.

17. A chimeric molecule comprising the TACIs polypeptide of claim 16 fused to a heterologous amino acid sequence.

18. The chimeric molecule of claim 17, wherein said heterologous amino acid sequence is an epitope tag sequence.

19. The chimeric molecule of claim 17, wherein said heterologous amino acid sequence is a Fc region of an immunoglobulin.

20. An isolated monoclonal antibody which binds to the TACIs polypeptide of claim 16.

21. A composition comprising the TACIs polypeptide of claim 16 and a carrier.

22. The composition of claim 21 wherein said carrier is a pharmaceutically-acceptable carrier.

23. An isolated nucleic acid comprising (a) DNA encoding a BR3 polypeptide comprising the sequence of amino acid residues 1 to 184 of SEQ ID NO:16, or (b) the complement of the DNA molecule of (a).

24. The nucleic acid of claim 23, wherein said DNA comprises the coding nucleotide sequence of SEQ ID NO:15.

25. The nucleic acid of claim 24, wherein said DNA consists of coding nucleotide sequence of SEQ ID NO:15.

26. An isolated nucleic acid comprising DNA which has (a) at least 95% sequence identity to the coding sequence of nucleotides of SEQ ID NO:15 and (b) encodes a BR3 polypeptide.

27. An isolated nucleic acid comprising DNA from the group consisting of:
a) a DNA having at least 90% sequence identity to a DNA sequence encoding a BR3 polypeptide comprising amino acid residues 1 to 184 of SEQ ID NO:16;
b) a DNA sequence, that hybridizes under stringent conditions to a DNA of a);
c) a DNA sequence that, due to the degeneracy of the genetic code, encodes a BR3 polypeptide of a); and
d) DNA fully complementary to the DNA of a), b), or c).

28. A vector comprising the nucleic acid of claim 27.

29. The vector of claim 28 operably linked to control sequences recognized by a host cell transformed with the vector.

30. A host cell which includes the vector of claim 28.

31. The host cell of claim 30, wherein said cell is a CHO cell.

32. The host cell of claim 30, wherein said cell is an E. coli.

33. The host cell of claim 30, wherein said cell is a yeast cell.

34. A process for producing a BR3 polypeptide comprising culturing the host cell of claim 30 under conditions suitable for expression of said BR3 polypeptide and recovering said BR3 polypeptide from the cell culture.

35. An isolated BR3 polypeptide comprising amino acid residues 1 to 184 of FIG. 6B (SEQ ID NO:16).

36. An isolated BR3 polypeptide comprising the sequence of contiguous amino acid residues 1 to 184 of FIG. 6B (SEQ ID NO:16).

37. An isolated soluble BR3 polypeptide comprising amino acid residues 1 to 77 or 2 to 62 of FIG. 6B (SEQ ID NO:16).

38. An isolated BR3 polypeptide comprising a polypeptide selected from the group consisting of:
a) a BR3 polypeptide comprising amino acid residues 1 to 184, 1 to 77, or 2 to 62 of FIG. 6B (SEQ ID NO:16) and
b) a fragment of a), wherein said fragment is a biologically active polypeptide.

39. A chimeric molecule comprising the BR3 polypeptide of claim 38 fused to a heterologous amino acid sequence.

40. The chimeric molecule of claim 39, wherein said heterologous amino acid sequence is an epitope tag sequence.

41. The chimeric molecule of claim 39, wherein said heterologous amino acid sequence is a Fc region of an immunoglobulin.

42. An isolated monoclonal antibody which binds to the BR3 polypeptide of claim 38.

43. A composition comprising the BR3 polypeptide of claim 38 and a carrier.

44. The composition of claim 43 wherein said carrier is a pharmaceutically-acceptable carrier.

45. A method of inhibiting or neutralizing TALL-1 polypeptide biological activity in mammalian cells, comprising exposing said mammalian cells to an effective amount of TALL-1 polypeptide antagonist, wherein said TALL-1 polypeptide antagonist is selected from the group consisting of
a) a TACIs receptor immunoadhesin;
b) a BR3 receptor immunoadhesin;
c) a TACIs receptor linked to a nonproteinaceous polymer selected from the group consisting of polyethylene glycol, polypropylene glycol, and polyoxyalkylene;
d) a BR3 receptor linked to a nonproteinaceous polymer selected from the group consisting of polyethylene glycol, polypropylene glycol, and polyoxyalkylene;
e) a TACIs receptor antibody; and
f) a BR3 receptor antibody.

46. The method of claim 45 wherein said TACIs receptor immunoadhesin comprises a TACIs extracellular domain sequence fused to a Fc region of an immunoglobulin.

47. The method of claim 45 wherein said BR3 receptor immunoadhesin comprises a BR3 extracellular domain sequence fused to a Fc region of an immunoglobulin.

48. The method of claim 45 wherein said TALL-1 polypeptide antagonist comprises an antagonist molecule which inhibits or neutralizes both TALL-1 polypeptide and APRIL polypeptide biological activity in mammalian cells.

49. The method of claim 45 wherein said mammalian cells comprise white blood cells.

50. A method of inhibiting or neutralizing APRIL polypeptide biological activity in mammalian cells, comprising exposing said mammalian cells to an effective amount of APRIL polypeptide antagonist, wherein said April polypeptide antagonist is selected front the group consisting of
a) a TACIs receptor immunoadhesin;
b) a TACIs receptor linked to a nonproteinaceous polymer selected from the group consisting of polyethylene glycol, polypropylene glycol, and polyoxyalkylene;
c) a TACIs receptor antibody.

51. The method of claim 50 wherein said TACIs receptor immunoadhesin comprises a TACIs extracellular domain sequence fused to a Fc region of an immunoglobulin.

52. The method of claim 50 wherein said APRIL polypeptide antagonist comprises an antagonist molecule which inhibits or neutralizes both TALL-1 polypeptide and APRIL polypeptide biological activity in mammalian cells.

53. The method of claim 50 wherein said mammalian cells comprise white blood cells.

54. A method of enhancing or stimulating TACI polypeptide activity in mammalian cells, comprising exposing said mammalian cells to an effective amount of TACIs polypeptide agonist, wherein said TACIs polypeptide agonist comprises an anti-TACIs agonist antibody.

55. A method of enhancing or stimulating BR3 polypeptide activity in mammalian cells, comprising exposing said mammalian cells to en effective amount of BR3 polypeptide agonist, wherein said BR3 polypeptide agonist comprises an anti-BR3 agonist antibody.

56. A method of treating systemic lupus erythmatosus in a mammal, comprising administering to said mammal an effective amount of BR3 receptor immunoadhesin which comprises a BR3 extracellular domain sequence fused to a Fc region of an immunoglobulin.

57. A method of conducting a screening assay to identify a candidate molecule which acts as an antagonist or agonist of TALL-1, TACI, TACIs, BCMA or BR3, comprising an assay using the TACIs DNA or polypeptide of claims 5 or 16, or the BR3 DNA or polypeptide of claims 27 or 38.
